(19) United States
(12) Patent Application Publication
(10) Pub. No.: US 2025/0304556 A1
(43) Pub. Date: Oct. 2, 2025

(54) PROCESSES FOR PREPARATION OF PRMT5 INHIBITORS AND INTERMEDIATES FOR MAKING THE SAME

(71) Applicant: MIRATI THERAPEUTICS, INC., PRINCETON, NJ (US)

(72) Inventors: MICHAL ACHMATOWICZ, SAN DIEGO, CA (US); JOSEPH LIZZA, HATFIELD, PA (US); GUIHUI CHEN, HATFIELD, PA (US); HONGLIANG CHEN, HATFIELD, PA (US)

(73) Assignee: MIRATI THERAPEUTICS, INC., PRINCETON, NJ (US)

(21) Appl. No.: 19/097,336

(22) Filed: Apr. 1, 2025

Related U.S. Application Data
(60) Provisional application No. 63/573,021, filed on Apr. 2, 2024.

(51) Int. Cl. C07D 403/04; C07C 51/04; C07C 63/70; C07D 231/12

(52) U.S. Cl. CPC C07D 403/04; C07C 51/04; C07C 63/70; C07D 231/12

(57) ABSTRACT

Disclosed are processes for preparation of Protein Arginine N-Methyl Transferase 5 (PRMT5) Inhibitors and intermediates for making the same.

### CROSS REFERENCE TO RELATED APPLICATIONS

This application claims the benefit of U.S. Provisional Application No. 63/573,021, filed Apr. 2, 2024, the entire content of which is hereby incorporated herein by reference.

### FIELD

The present disclosure relates generally to processes for preparation of Protein Arginine N-Methyl Transferase 5 (PRMT5) Inhibitors and intermediates for making the same.

### BACKGROUND

Protein Arginine N-Methyl Transferase (PRMT5) is a Type II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with MEP50 (methylosome protein 50), which is required for substrate recognition and orientation and is also required for PRMT5-catalyzed histone 2A and histone 4 methyltransferase activity. See, e.g., Ho et al., (2013) PLOS ONE 8(8): 10.1371/annotation/e6b5348e-9052-44ab-8f06-90d01dc88fc2. Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene. See, e.g., Firestone & Schramm (2017) J. Am. Chem Soc. 139(39):13754-13760.doi: 10.1021/jacs.7b05803. Epub 2017 Sep. 20.

Cells lacking MTAP activity have elevated levels of the MTAP substrate methylthioadenosine (MTA), which is a potent inhibitor of PRMT5. Inhibition of PRMT5 activity results in reduced methylation activity and increased sensitivity of cellular proliferation to PRMT5 depletion or loss of activity. Hence, the loss of MTAP activity reduces methylation activity of PRMT5 making the cells selectively dependent on PRMT5 activity.

Thus, MTA-cooperative inhibition of PRMT5 activity in MTAP deleted cancers can provide therapeutic benefit for a wide range of cancers. The compounds of the invention provide this therapeutic benefit as MTA-cooperative inhibitors of PRMT5 that negatively modulate the activity of MTA-bound PRMT5 in a cell, particularly an MTAP-deficient cell, or for treating various forms of MTAP-associated cancer.

In particular, 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (MRTX-1719) is a potent and selective inhibitor of PRMT5 and has been found to be pharmacologically active. Thus, synthetic methods for producing this and similar compounds on a commercial scale are necessary.

### SUMMARY

Synthetic methods are disclosed that are useful for preparing various 2-(4-(1-oxo-1,2-dihydrophthalazin-6-yl)-1H-pyrazol-5-yl)benzonitriles, including MRTX-1719, which is 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile and has the following structure:

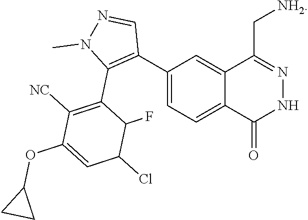 [SMILES: Cn1ncc(-c2ccc3c(=O)[nH]nc(CN)c3c2)c1C1=C(C#N)C(OC2CC2)=CC(Cl)C1F]

In one aspect, the present disclosure provides processes for preparing compounds of Formula (I)

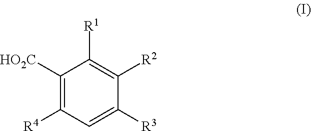

wherein:
R¹ is halogen, an aryl group, or a heteroaryl group;
each of R² and R³ is independently halogen; and
R⁴ is halogen, C₁-C₆ alkyl, C₃-C₆ cycloalkyl, halo C₁-C₆ alkyl, or OR⁵, wherein R⁵ is C₁-C₆ alkyl, C₃-C₆ cycloalkyl, or halo C₁-C₆ alkyl,
the process comprising treating a compound of Formula (IA)

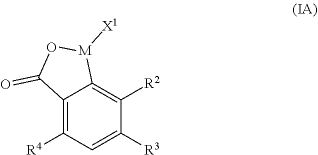

with an electrophile which is I₂, Br₂, Cl₂, an aryl compound, or a heteroaryl compound,
wherein:
   M represents a divalent metal of a divalent metal base; and
   X¹ represents a counterion of the divalent metal base.

In another aspect, the present disclosure provides processes for preparing compounds of Formula (II)

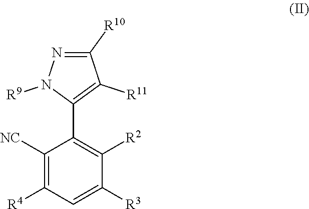

wherein:
each of R², R³, and R¹¹ is independently halogen;
R⁴ is halogen, C₁-C₆ alkyl, C₃-C₆ cycloalkyl, halo C₁-C₆ alkyl, or OR⁵, wherein R⁵ is C₁-C₆ alkyl, C₃-C₆ cycloalkyl, or halo C₁-C₆ alkyl;
R⁹ is C₁-C₆ alkyl, C₃-C₆ cycloalkyl, or halo C₁-C₆ alkyl; and
R¹⁰ is hydrogen,
   the process comprising:
(a) reacting a compound of Formula (I)

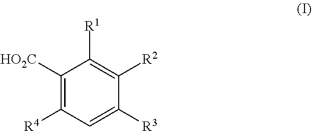

with an amine to provide a compound of Formula (IIA)

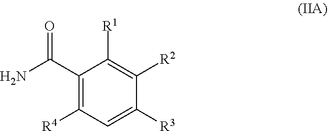

wherein R¹ is halogen, an aryl group, or a heteroaryl group;
(b) dehydrating the compound of Formula (IIA) to provide a compound of Formula (IIB)

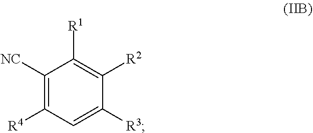

(c) coupling the compound of Formula (IIB) with an organoboron compound or an organozinc compound to provide a compound of Formula (IIC)

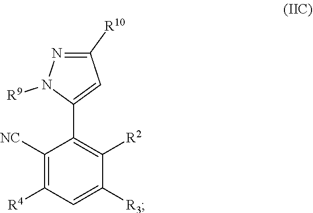

and
(d) halogenating the compound of Formula (IIC) to provide the compound of Formula (II).

In another aspect, the present disclosure provides processes for preparing a compound of Formula (III)

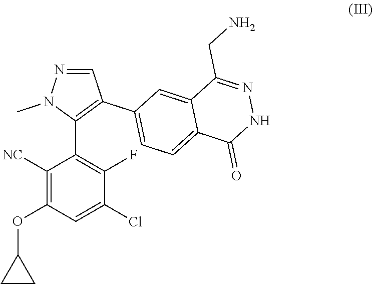 [SMILES: Cn1ncc(-c2ccc3c(=O)[nH]nc(CN)c3c2)c1-c1c(F)c(Cl)cc(OC2CC2)c1C#N]

the process comprising:
(a) treating 4-chloro-2,5-difluorobenzoic acid with a divalent metal base to provide a mixture;
(b) treating the mixture with iodine to provide 4-chloro-3,6-difluoro-2-iodobenzoic acid;
(c) reacting the 4-chloro-3,6-difluoro-2-iodobenzoic acid with an amine to provide 4-chloro-3,6-difluoro-2-iodobenzamide;
(d) dehydrating the 4-chloro-3,6-difluoro-2-iodobenzamide to provide 4-chloro-3,6-difluoro-2-iodobenzonitrile;
(e) coupling the 4-chloro-3,6-difluoro-2-iodobenzonitrile with an organoboron compound to provide 4-chloro-3,6-difluoro-2-(1-methyl-1H-pyrazol-5-yl)benzonitrile;
(f) iodinating the 4-chloro-3,6-difluoro-2-(1-methyl-1H-pyrazol-5-yl)benzonitrile to provide 4-chloro-3,6-difluoro-2-(4-iodo-1-methyl-1H-pyrazol-5-yl)benzonitrile; and
(g) reacting the 4-chloro-3,6-difluoro-2-(4-iodo-1-methyl-1H-pyrazol-5-yl)benzonitrile with cyclopropanol to provide 4-chloro-6-cyclopropoxy-3-fluoro-2-(4-iodo-1-methyl-1H-pyrazol-5-yl)benzonitrile.

In another aspect, the present disclosure provides compounds of Formula (IV)

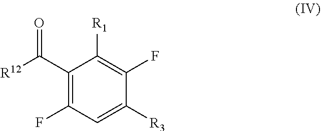

wherein:
R¹ is iodo, an aryl group, or a heteroaryl group;
R³ is bromo or chloro; and
R¹² is-OR¹³ or —NR¹⁴R¹⁵, wherein each of R¹³, R¹⁴, and R¹⁵ is independently hydrogen, C₁-C₆ alkyl, or C₃-C₆ cycloalkyl.

In another aspect, the present disclosure provides compositions comprising a mixture of 4-chloro-2,5-difluorobenzoic acid and 4-chloro-3,6-difluoro-2-iodobenzoic acid.

Other aspects and embodiments of the disclosure are evident in view of the detailed description provided herein.

### DETAILED DESCRIPTION OF THE INVENTION

The present invention relates to processes for preparation of PRMT5 Inhibitors and intermediates for making the same.

### Definitions

Unless defined otherwise, all technical and scientific terms used herein have the same meaning as is commonly understood by one of skill in the art to which this invention belongs. All patents, patent applications, and publications referred to herein are incorporated by reference to the extent they are consistent with the present disclosure. Terms and ranges have their generally defined definition unless expressly defined otherwise.

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g., CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which a divalent moiety is required and is stated as being “aryl,” those skilled in the art will understand that the term “aryl” refers to the corresponding divalent moiety, arylene. All atoms are understood to have their normal number of valences for bond formation (i.e., 4 for carbon, 3 for N, 2 for O, and 2, 4, or 6 for S, depending on the oxidation state of the S).

The term “amino” refers to —NH₂.

The term “acetyl” refers to —C(O) CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁, and C₁₂ groups. Alkyl groups may be branched or unbranched. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁, and C₁₂ groups.

Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁, and C₁₂ groups.

Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Examples of alkenylene groups include, without limitation, ethenylene, propenylene, and butenylene. Examples of alkynylene groups include, without limitation, ethynylene, propynylene, and butynylene.

The term “alkoxy” refers to —O(C₁-C₆ alkyl).

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁, and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “C₃-C₆ cycloalkyloxy” refers to groups of the formula —O(C₃-C₆ cycloalkyl).

The term “heteroalkyl” refers to an alkyl group, as defined hereinabove, wherein one or more carbon atoms in the chain are independently replaced O, S, or NRK, wherein Rx is hydrogen or C₁-C₃ alkyl. Examples of heteroalkyl groups include methoxymethyl, methoxyethyl, and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. A representative aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An representative aralkyl group is —(C₁-C₆)alkyl(C₆-C₁₀) aryl, including, without limitation, benzyl, phenethyl, and naphthylmethyl. For example, an arC₁-C₃alkyl is an aryl group covalently linked to a C₁-C₃ alkyl.

A “heterocyclyl” or “heterocyclic” or “heterocycloalkyl” group is a mono- or bicyclic (e.g., fused) ring structure having from 3 to 12 atoms, (3, 4, 5, 6, 7, 8, 9, 10, 11 or 12 atoms), for example 4 to 8 atoms, wherein one or more ring atoms are independently N, O, or S, and the remainder of the ring atoms are quaternary or carbonyl carbons. Examples of heterocyclic groups include, without limitation, epoxy, oxiranyl, oxetanyl, azetidinyl, aziridinyl, tetrahydrofuranyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. The heterocyclic groups can be attached to a parent group (i.e., the point of attachment) via any ring atom, including one of the heteroatoms or one of the carbon atoms, in the heterocyclic ring group. As chemically required, the heterocyclic ring may be attached to one or more other groups, for instance if operating as a bridging group. The term “heterocyclyl” also includes fused multicyclic (e.g., bicyclic) ring systems in which one or more of the fused rings is aromatic or non-aromatic, provided that at least one ring is non-aromatic contains an N, O, or S ring atom. Examples of such fused multicyclic ring systems are indolinyl, indolin-2-yl, 2,3-dihydrobenzofuran-2-yl, and 2,3,4,5-tetrahydrobenzo[d]oxazol-2-yl. Each of these examples is a 9-membered heterocyclyl.

As used herein, the term “heteroaryl” refers to a group having 5 to 14 ring atoms, preferably 5, 6, 10, 13, or 14 ring atoms; having 6, 10, or 14 π electrons shared in a cyclic array; and having, in addition to carbon atoms, from one to three heteroatoms that are each independently N, O, or S. “Heteroaryl” also includes fused multicyclic (e.g., bicyclic) ring systems in which one or more of the fused rings is non-aromatic, provided that at least one ring is aromatic and at least one ring contains an N, O, or S ring atom. The heteroaryl groups can be attached to a parent group (i.e., the point of attachment) via any ring atom, including one of the heteroatoms or one of the carbon atoms, in the heteroaryl ring group. As chemically required, the heteroaryl may be attached to one or more other groups, for instance if operating as a bridging group.

Examples of heteroaryl groups include acridinyl, azocinyl, benzimidazolyl, benzofuranyl, benzo[d]oxazol-2 (3H)-one, 2H-benzo[b][1,4]oxazin-3 (4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Preferred heteroaryl groups have 5-10 members. Other preferred heteroaryl groups have from 5-6 members.

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents, it is meant that the group optionally has multiple non-hydrogen substituents, for example, from one to five, or from one to four, or from one to three, or one, or two non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Representative haloalkyls are trifluoromethyl, difluoromethyl, fluorochloromethyl, chloromethyl, and fluoromethyl.

The term “hydroxyalkyl” refers to -alkylene-OH.

It is to be understood that each individual atom present in Formula (I) and the compounds within Formula (I), may be present in the form of any of its naturally occurring isotopes, with the most abundant isotope(s) being preferred. Thus, by way of example, each individual hydrogen atom present in Formula (I), or in the formulae depicted hereinafter, may be present as a ¹H, ²H (deuterium; D), or ³H (tritium; T) atom, preferably ¹H. Similarly, by way of example, each individual carbon atom present in formula (I), or in the formulae depicted hereinafter, may be present as a ¹²C, ¹³C, or ¹⁴C atom, preferably ¹²C.

As used herein, “an effective amount” of a compound is an amount that is sufficient to treat an inflammatory condition.

As used herein, a “therapeutically effective amount” of a compound is an amount that is sufficient to ameliorate or in some manner reduce a symptom or stop or reverse progression of an inflammatory condition. Such amount may be administered as a single dosage or may be administered according to a regimen, whereby it is effective.

As used herein, “treatment” means any manner in which the symptoms or pathology of a condition, disorder or disease in a patient are ameliorated or otherwise beneficially altered.

As used herein, “amelioration of the symptoms of an inflammatory condition by administration of a particular compound or pharmaceutical composition” refers to any lessening, whether permanent or temporary, lasting or transient, that can be attributed to or associated with administration of the composition.

As used herein, the terms “individual,” “patient,” or “subject” are used interchangeably, refers to any animal, including mammals, preferably humans.

### Processes of Preparation

In one aspect, the present disclosure provides processes for preparing compounds of Formula (I)

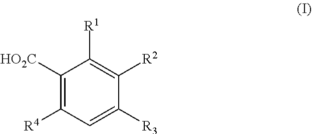

wherein:
R¹ is halogen, an aryl group, or a heteroaryl group;
each of R² and R³ is independently halogen; and
R⁴ is halogen, C₁-C₆ alkyl, C₃-C₆ cycloalkyl, halo C₁-C₆ alkyl, or OR⁵, wherein R⁵ is C₁-C₆ alkyl, C₃-C₆ cycloalkyl, or halo C₁-C₆ alkyl,
   the process comprising treating a compound of Formula (IA)

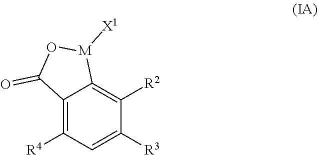

with an electrophile which is I₂, Br₂, Cl₂, an aryl compound, or a heteroaryl compound,
wherein:
   M represents a divalent metal of a divalent metal base; and
   X¹ represents a counterion of the divalent metal base.

In certain embodiments of the process for preparing the compounds of Formula (I) as otherwise described herein, R¹ is halogen.

In certain embodiments of the process for preparing the compounds of Formula (I) as otherwise described herein, R¹ is iodo.

In certain embodiments of the process for preparing the compounds of Formula (I) as otherwise described herein, R¹ is an aryl group.

In certain embodiments of the process for preparing the compounds of Formula (I) as otherwise described herein, R¹ is a C₆-C₁₀ aryl group optionally substituted with one or more of C₁-C₆ alkyl, halo C₁-C₆ alkyl, C₃-C₆ cycloalkyl, or C₁-C₆ alkoxy.

In certain embodiments of the process for preparing the compounds of Formula (I) as otherwise described herein, the aryl group is phenyl, naphthyl, anthracenyl, fluorenyl, benzyl, phenethyl, or naphthylmethyl.

In certain embodiments of the process for preparing the compounds of Formula (I) as otherwise described herein, R¹ is a heteroaryl group.

In certain embodiments of the process for preparing the compounds of Formula (I) as otherwise described herein, R¹ is a 5- or 6-member heteroaryl group optionally substituted with one or more of C₁-C₆ alkyl, halo C₁-C₆ alkyl, C₃-C₆ cycloalkyl, or C₁-C₆ alkoxy.

In certain embodiments of the process for preparing the compounds of Formula (I) as otherwise described herein, the heteroaryl group is furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, or triazinyl.

In certain embodiments of the process for preparing the compounds of Formula (I) as otherwise described herein, the electrophile is a heteroaryl compound of Formula (IB)

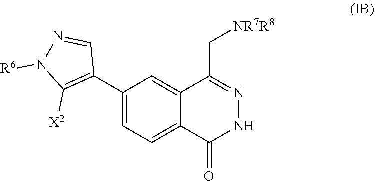

wherein:
X² is I, Br, or CI;
R⁶ is hydrogen, C₁-C₆ alkyl, C₃-C₆ cycloalkyl, or halo C₁-C₆ alkyl; and
each of R⁷ and R⁸ is independently hydrogen or a protecting group, or R⁷ and R⁸ together with the N to which they bind form a ring structure.

In certain embodiments of the process for preparing the heteroaryl compound of Formula (IB) as otherwise described herein, R⁶ is methyl.

In certain embodiments of the process for preparing the heteroaryl compound of Formula (IB) as otherwise described herein, each of R⁷ and R⁸ is independently hydrogen.

In certain embodiments of the process for preparing the heteroaryl compound of Formula (IB) as otherwise described herein, one of R⁷ and R⁸ is hydrogen, and the other is a protecting group.

In certain embodiments of the process for preparing the heteroaryl compound of Formula (IB) as otherwise described herein, the protecting group is a tert-butyloxycarbonyl (Boc) group.

In certain embodiments of the process for preparing the heteroaryl compound of Formula (IB) as otherwise described herein, the ring structure is a phthalimide group.

In certain embodiments of the process for preparing the compounds of Formula (I) as otherwise described herein, R² is fluoro.

In certain embodiments of the process for preparing the compounds of Formula (I) as otherwise described herein, R³ is chloro.

In certain embodiments of the process for preparing the compounds of Formula (I) as otherwise described herein, R³ is bromo.

In certain embodiments of the process for preparing the compounds of Formula (I) as otherwise described herein, R⁴ is fluoro.

In certain embodiments of the process for preparing the compounds of Formula (I) as otherwise described herein, R⁴ is OR⁵, and wherein R⁵ is cyclopropyl.

In certain embodiments of the process for preparing the compounds of Formula (I) as otherwise described herein, the compound of Formula (IA) is provided in a solvent. Suitable solvents include an ethereal solvent and an aprotic solvent. In certain embodiments as otherwise described herein, the solvent is dimethyl ether, diethyl ether, tetrahydrofuran (THF), 2-methyltetrahydrofuran (2-MeTHF), 1-4-dioxane, 1,2-dimethoxyethane, methyl tert-butyl ether (MTBE), cyclopentyl methyl ether (CPME), toluene, or acetonitrile.

certain embodiments of the process for preparing the compounds of Formula (I) as otherwise described herein, the compound of Formula (IA) is provided in THF or 2-MeTHF. In certain embodiments, the process for preparing the compounds of Formula (I) is carried out in a solvent. Suitable solvents include an ethereal solvent and an aprotic solvent. In certain embodiments as otherwise described herein, the solvent is dimethyl ether, diethyl ether, THF, 2-MeTHF, 1-4-dioxane, 1,2-dimethoxyethane, MTBE, CPME, toluene, or acetonitrile.

In certain embodiments of the process for preparing the compounds of Formula (I) as otherwise described herein, the treating of the compound of Formula (IA) with the electrophile is carried out in THF or 2-MeTHF.

In certain embodiments, the process for preparing the compounds of Formula (I) is carried out under an inert atmosphere. Suitable gases for producing an inert atmosphere include nitrogen and argon.

In certain embodiments of the process for preparing the compounds of Formula (I) as otherwise described herein, the treating of the compound of Formula (IA) with the electrophile is carried out under a nitrogen atmosphere.

In certain embodiments, the process for preparing the compounds of Formula (I) is carried out under an inert atmosphere at a temperature in a range of about −10 to about −30° C. Representative temperatures for this reaction are about −10° C., about −15° C., about −20° C., about −25° C., and about −30° C.

In certain embodiments of the process for preparing the compounds of Formula (I) as otherwise described herein, the treating of the compound of Formula (IA) with the electrophile is carried out under a nitrogen atmosphere at a temperature of about −25° C.

In certain embodiments, the process for preparing the compounds of Formula (I) is carried out for at least 0.5 hour. Representative times for completion of this reaction are about 1 hour, about 1.5 hours, and about 2 hours.

In certain embodiments of the process for preparing the compounds of Formula (I) as otherwise described herein, the treating of the compound of Formula (IA) with the electrophile is carried out for about 1 hour.

In certain embodiments of the process for preparing the compounds of Formula (I) as otherwise described herein, the compound of Formula (IA) is prepared by treating a compound of Formula (IC)

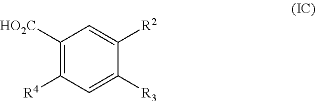

with the divalent metal base M-X¹.

In certain embodiments, the preparation of the compound of Formula (IA) is carried out in a solvent. Suitable solvents include an ethereal solvent and an aprotic solvent. In certain embodiments as otherwise described herein, the solvent is dimethyl ether, diethyl ether, THF, 2-MeTHF, 1-4-dioxane, 1,2-dimethoxyethane, MTBE, CPME, toluene, or acetonitrile.

In certain embodiments of the process for preparing the compound of Formula (IA) as otherwise described herein, the treatment of the compound of Formula (IC) with the divalent metal base is carried out in THF or 2-MeTHF.

In certain embodiments, the preparation of the compound of Formula (IA) is carried out under an inert atmosphere. Suitable gases for producing an inert atmosphere include nitrogen and argon.

In certain embodiments of the process for preparing the compound of Formula (IA) as otherwise described herein, the treatment of the compound of Formula (IC) with the divalent metal base is carried out under a nitrogen atmosphere.

In certain embodiments, the preparation of the compound of Formula (IA) is carried out under an inert atmosphere at a temperature of about −30° C. or higher. Representative temperatures for this reaction are about −30° C., about −25° C., about −20° C., about −15° C., and about −10° C.

In certain embodiments of the process for preparing the compound of Formula (IA) as otherwise described herein, the treatment of the compound of Formula (IC) with the divalent metal base is carried out under a nitrogen atmosphere at a temperature of about −20° C.

In certain embodiments, the preparation of the compound of Formula (IA) is carried out for at least 1 hour. Representative times for completion of this reaction are about 2 hours, about 3 hours, and about 4 hours.

In certain embodiments of the process for preparing the compound of Formula (IA) as otherwise described herein, the treatment of the compound of Formula (IC) with the divalent metal base is carried out for about 3 hours.

In certain embodiments, the preparation of the compound of Formula (IA) is carried out with a molar ratio of the compound of Formula (IC) to the divalent metal base of about 2.0 to 1.0, or about 2.2 to 1.0, or about 2.5 to 1.0.

In certain embodiments of the process for preparing the compound of Formula (IA) as otherwise described herein, the treatment of the compound of Formula (IC) with the divalent metal base is carried out with a molar ratio of the compound of Formula (IC) to the divalent metal base of about 2.2 to 1.0.

In certain embodiments of the process for preparing the compounds of Formula (I) as otherwise described herein, the divalent metal base is a magnesium base. In certain embodiments, the magnesium base is an alkyl Grignard reagent having a formula of R^{G}MgX, wherein R^{G} is C₁-C₆ alkyl, C₃-C₆ cycloalkyl, or NR¹R², and wherein each of R¹ and R² is independently C₁-C₆ alkyl, C₃-C₆ cycloalkyl, or together with the nitrogen to which they are attached form a 3-8 membered monocyclic heterocyclyl group, and wherein the 3-8 monocyclic heterocyclyl group is optionally substituted with C₁-C₆ alkyl, and wherein X is I, Br, or C₁. Suitable magnesium bases include TMPMgCl·LiCl, (TMP)₂Mg·2LiCl, and i-PrMgCl·LiCl, and wherein TMP is 2,2,6,6-tetramethylpiperidyl.

In certain embodiments of the process for preparing the compounds of Formula (I) as otherwise described herein, the magnesium base is supplied to the reaction for treating the compound of Formula (IC) in a solvent. Suitable solvents include dimethyl ether, diethyl ether, THF, 2-MeTHF, 1-4-dioxane, 1,2-dimethoxyethane, MTBE, CPME, toluene, and acetonitrile. In certain embodiments of the process for preparing the compounds of Formula (I) as otherwise described herein, the divalent metal base is a zinc base. Suitable zinc bases include (TMP)₂Zn·2LiCl and TMPZnCl·LiCl, and wherein TMP is 2,2,6,6-tetramethylpiperidyl.

In certain embodiments of the process for preparing the compounds of Formula (I) as otherwise described herein, the zinc base is dialkylzinc or dialkylzinc halide.

In certain embodiments of the process for preparing the compounds of Formula (I) as otherwise described herein, the zinc base is Et₂Zn or EtZnCl.

In certain embodiments of the process for preparing the compounds of Formula (I) as otherwise described herein, the compound of Formula (I) is 4-chloro-3,6-difluoro-2-iodobenzoic acid, which is prepared by:
(a) treating 4-chloro-2,5-difluorobenzoic acid with the divalent metal base to provide a mixture; and
(b) treating the mixture with iodine to provide 4-chloro-3,6-difluoro-2-iodobenzoic acid.

In one aspect, the present disclosure provides processes for preparing compounds of Formula (II)

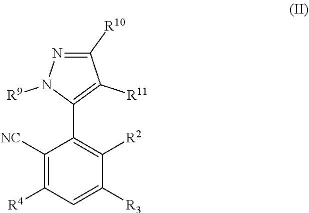

wherein:
each of R², R³, and R¹¹ is independently halogen;
R⁴ is halogen, C₁-C₆ alkyl, C₃-C₆ cycloalkyl, halo C₁-C₆ alkyl, or OR⁵, wherein R⁵ is C₁-C₆ alkyl, C₃-C₆ cycloalkyl, or halo C₁-C₆ alkyl;
R⁹ is C₁-C₆ alkyl, C₃-C₆ cycloalkyl, or halo C₁-C₆ alkyl; and
R¹⁰ is hydrogen,
   the process comprising:
(a) reacting a compound of Formula (I)

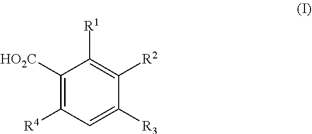

with an amine to provide a compound of Formula (IIA)

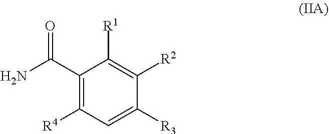

wherein R¹ is halogen, an aryl group, or a heteroaryl group;
(b) dehydrating the compound of Formula (IIA) to provide a compound of Formula (IIB)

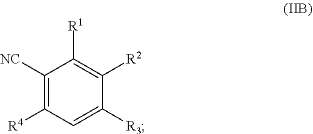

(c) coupling the compound of Formula (IIB) with an organoboron compound to provide a compound of Formula (IIC)

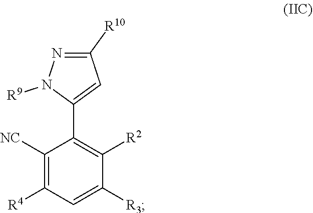

and
(d) halogenating the compound of Formula (IIC) to provide the compound of Formula (II).

In certain embodiments of the process for preparing the compounds of Formula (II) as otherwise described herein, R² is fluoro.

In certain embodiments of the process for preparing the compounds of Formula (II) as otherwise described herein, R³ is chloro.

In certain embodiments of the process for preparing the compounds of Formula (II) as otherwise described herein, R³ is bromo.

In certain embodiments of the process for preparing the compounds of Formula (II) as otherwise described herein, R⁴ is fluoro.

In certain embodiments of the process for preparing the compounds of Formula (II) as otherwise described herein, R⁴ is OR⁵, and wherein R⁵ is cyclopropyl.

In certain embodiments of the process for preparing the compounds of Formula (II) as otherwise described herein, R⁹ is C₁-C₆ alkyl.

In certain embodiments of the process for preparing the compounds of Formula (II) as otherwise described herein, R⁹ is methyl.

In certain embodiments of the process for preparing the compounds of Formula (II) as otherwise described herein, R¹⁰ is hydrogen.

In certain embodiments of the process for preparing the compounds of Formula (II) as otherwise described herein, R¹¹ is iodo.

In certain embodiments of the process for preparing the compounds of Formula (II) as otherwise described herein, step (a) further comprises treating the compound of Formula (I) with an activating reagent, followed by reacting with the amine to provide the compound of Formula (IIA).

In certain embodiments of the process for preparing the compounds of Formula (IIA) as otherwise described herein, the activating reagent is carbonyl diimidazole (CDI), 1,1′-thiocarbonyldiimidazole (TCDI), carbonyl dichloride, or thionyl chloride (SOCl₂).

In certain embodiments, the treating of the compound of Formula (I) with the activating reagent is carried out with a molar ratio of the compound of Formula (I) to the activating reagent of about 1 to 1.5, or about 1 to 2, or about 1 to 2.5.

In certain of the process for preparing the compounds of Formula (II) as otherwise described herein, in step (a), the treating of the compound of Formula (I) with the activating reagent is carried out with a molar ratio of the compound of Formula (I) to the activating reagent of about 1 to 2.

In certain embodiments of the process for preparing the compounds of Formula (II) as otherwise described herein, in step (a), the compound of Formula (I) is prepared by treating a compound of Formula (IA)

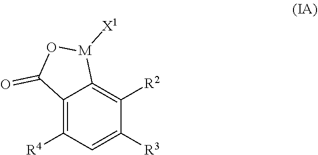

with an electrophile which is I₂, Br₂, Cl₂, an aryl compound, or a heteroaryl compound, wherein:
M represents a divalent metal of a divalent metal base; and
X¹ represents a counterion of the divalent metal base.

In certain embodiments of the process for preparing the compound of Formula (II) as otherwise described herein, in step (a), the compound of Formula (IA) is prepared by treating a compound of Formula (IB)

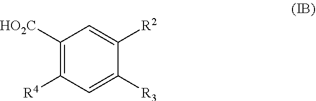

with the divalent metal base M-X¹.

In certain embodiments of the process for preparing the compounds of Formula (II) as otherwise described herein, R¹ is iodo.

In certain embodiments of the process for preparing the compounds of Formula (II) as otherwise described herein, the compound of Formula (II) is 4-chloro-3,6-difluoro-2-(4-iodo-1-methyl-1H-pyrazol-5-yl)benzonitrile, which is prepared by:
(a) reacting 4-chloro-3,6-difluoro-2-iodobenzoic acid with an amine to provide 4-chloro-3,6-difluoro-2-iodobenzamide;
(b) dehydrating 4-chloro-3,6-difluoro-2-iodobenzamide to provide 4-chloro-3,6-difluoro-2-iodobenzonitrile;
(c) coupling 4-chloro-3,6-difluoro-2-iodobenzonitrile with an organoboron compound to provide 4-chloro-3,6-difluoro-2-(1-methyl-1H-pyrazol-5-yl)benzonitrile; and
(b) iodinating 4-chloro-3,6-difluoro-2-(1-methyl-1H-pyrazol-5-yl)benzonitrile to provide 4-chloro-3,6-difluoro-2-(4-iodo-1-methyl-1H-pyrazol-5-yl)benzonitrile.

In certain embodiments of the process for preparing the compounds of Formula (II) as otherwise described herein, step (a) further comprises treating 4-chloro-3,6-difluoro-2-iodobenzoic acid with an activating reagent, followed by reacting with the amine to provide 4-chloro-3,6-difluoro-2-iodobenzamide.

In certain embodiments of the process for preparing the compounds of Formula (II) as otherwise described herein, the activating reagent is CDI, TCDI, carbonyl dichloride, or SOCl₂.

In certain embodiments of the process for preparing the compounds of Formula (II) as otherwise described herein, in step (a), 4-chloro-3,6-difluoro-2-iodobenzoic acid is prepared by:
(1) treating 4-chloro-2,5-difluorobenzoic acid with a divalent metal base to provide a mixture; and
(b) treating the mixture with iodine to provide 4-chloro-3,6-difluoro-2-iodobenzoic acid.

In certain embodiments of the process for preparing the compounds of Formula (II) as otherwise described herein, in step (a), the amine is ammonia or ammonium hydroxide.

In certain embodiments, the process for preparing the compound of Formula (IIA) is carried out in a solvent. Suitable solvents include an ethereal solvent and an aprotic solvent. In certain embodiments as otherwise described herein, the solvent is dimethyl ether, diethyl ether, THF, 2-MeTHF, 1-4-dioxane, 1,2-dimethoxyethane, MTBE, CPME, toluene, or acetonitrile.

In certain embodiments of the process for preparing the compounds of Formula (II) as otherwise described herein, step (a) is carried out in a solution of THF or 2-MeTHF.

In certain embodiments, the process for preparing the compound of Formula (IIA) is carried out under an inert atmosphere at a temperature in a range of about 20 to about 60° C. Suitable gases for producing an inert atmosphere include nitrogen and argon. Representative temperatures for this reaction are about 50° C. and about 55° C.

In certain embodiments of the process for preparing the compounds of Formula (II) as otherwise described herein, step (a) is carried out under a nitrogen atmosphere at a temperature of about 50° C.

In certain embodiments, the process for preparing the compound of Formula (IIA) is carried out for at least 0.5 hour. Representative times for completion of this reaction are at least 1 hour, or at least 1.5 hours, and at least 2 hours.

In certain embodiments of the process for preparing the compounds of Formula (II) as otherwise described herein, step (a) is carried out for at least 1 hour.

In certain embodiments of the process for preparing the compounds of Formula (II) as otherwise described herein, step (b) is carried out in the presence of trifluoroacetic anhydride (TFAA), phosphoryl chloride (POCl₃), SOCl₂, or oxalyl chloride (COCl)₂.

In certain embodiments, the process for preparing the compound of Formula (IIB) is carried out in a solvent. Suitable solvents include an ethereal solvent and an aprotic solvent. In certain embodiments as otherwise described herein, the solvent is dimethyl ether, diethyl ether, THF, 2-MeTHF, 1-4-dioxane, 1,2-dimethoxyethane, MTBE, CPME, toluene, or acetonitrile.

In certain embodiments of the process for preparing the compounds of Formula (II) as otherwise described herein, step (b) is carried out in a solution of THF or 2-MeTHF.

In certain embodiments, the process for preparing the compound of Formula (IIB) is carried out under an inert atmosphere at a temperature in a range of about 0 to about 25° C. Suitable gases for producing an inert atmosphere include nitrogen and argon. Representative temperatures for this reaction are about 0° C., about 10° C., and about 20° C.

In certain embodiments of the process for preparing the compounds of Formula (II) as otherwise described herein, step (b) is carried out under a nitrogen atmosphere at a temperature of about 10° C.

In certain embodiments, the process for preparing the compound of Formula (IIB) is carried out for at least 20 minutes. Representative times for completion of this reaction are about 0.5 hour and about 1 hour.

In certain embodiments of the process for preparing the compounds of Formula (II) as otherwise described herein, step (b) is carried out for about 0.5 hour.

In certain embodiments of the process for preparing the compound of Formula (II) as otherwise described herein, in step (c), the organoboron compound is a boronic acid or ester having a structure of Formula (IID)

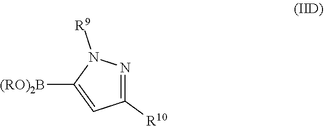

wherein R is hydrogen or C₁-C₆ alkyl.

In certain embodiments of the process for preparing the compound of Formula (IIC) as otherwise described herein, the organoboron compound is

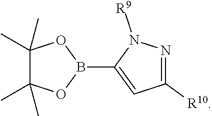

In certain embodiments of the process for preparing the compound of Formula (IIC) as otherwise described herein, the organozinc compound has a structure of Formula (IIE)

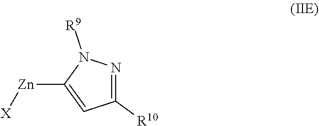

wherein X is I, Br, or C₁.

In certain embodiments of the process for preparing the compound of Formula (IIC) as otherwise described herein, step (c) is carried out in the presence of a catalyst.

In certain embodiments of the process for preparing the compound of Formula (IIC) as otherwise described herein, the catalyst is a palladium catalyst.

In certain embodiments of the process for preparing the compound of Formula (IIC) as otherwise described herein, the palladium catalyst is [P(t-Bu)₂(n-Bu)]—Pd-G3, Pd(amphos)Cl, or (amphos)₂PdCl₂.

In certain embodiments of the process for preparing the compound of Formula (IIC) as otherwise described herein, the palladium catalyst is a pre-catalyst prepared from a palladium source with a ligand.

In certain embodiments of the process as for preparing the compound of Formula (IIC) otherwise described herein, the palladium source is Pd(OAc)₂ or Pd₂(dba)₃.

In certain embodiments of the process as for preparing the compound of Formula (IIC) otherwise described herein, the ligand is amphos or P(tBu)₂(nBu).

In certain embodiments of the process as for preparing the compound of Formula (IIC) otherwise described herein, the catalyst is a nickel catalyst.

In certain embodiments, the process for preparing the compounds of Formula (IIC) is carried out with a molar ratio of the compound of Formula (IIB) to the organoboron compound or the organozinc compound of about 1 to 1.10, or about 1 to 1.12, or about 1 to 1.15, or about 1 to 1.18, or about 1 to 2.

In certain embodiments of the process for preparing the compounds of Formula (II) as otherwise described herein, in step (c), a molar ratio of the compound of Formula (IIB) to the organoboron compound or the organozinc compound is about 1 to 1.15.

In certain embodiments, the process for preparing the compound of Formula (IIC) is carried out in a solvent. Suitable solvents include an ethereal solvent and an aprotic solvent. In certain embodiments as otherwise described herein, the solvent is dimethyl ether, diethyl ether, THF, 2-MeTHF, 1-4-dioxane, 1,2-dimethoxyethane, MTBE, CPME, toluene, or acetonitrile.

In certain embodiments of the process for preparing the compound of Formula (IIC) as otherwise described herein, step (c) is carried out in a solution of THF or toluene.

In certain embodiments, the process for preparing the compound of Formula (IIC) is carried out under an inert atmosphere at a temperature in a range of about 60 to about 80° C. Suitable gases for producing an inert atmosphere include nitrogen and argon. Representative temperatures for this reaction are about 65° C., about 70° C., and about 75° C.

In certain embodiments of the process for preparing the compound of Formula (IIC) as otherwise described herein, step (c) is carried out under a nitrogen atmosphere at a temperature of about 75° C.

In certain embodiments, the process for preparing the compound of Formula (IIC) is carried out for at least 15 hour. Representative times for completion of this reaction are about 18 hours, about 20 hours, and about 22 hours.

In certain embodiments of the process for preparing the compounds of Formula (IIC) as otherwise described herein, step (c) is carried out for about 20 hours.

In certain embodiments of the process for preparing the compounds of Formula (II) as otherwise described herein, step (d) comprises iodinating the compound of step (c) to provide the compound of Formula (II).

In certain embodiments of the process for preparing the compounds of Formula (II) as otherwise described herein, step (d) is carried out in the presence of an iodinating reagent.

In certain embodiments of the process for preparing the compounds of Formula (II) as otherwise described herein, the iodinating reagent is molecular iodine or N-iodosuccinimide (NIS).

In certain embodiments, the halogenation of the compound of Formula (IIC) is carried out with a molar ratio of the compound of Formula (IIC) to the iodinating compound of about 1 to 1, or about 1 to 1.1, or about 1 to 1.2.

In certain embodiments of the process for preparing the compounds of Formula (II) as otherwise described herein, in step (d), a molar ratio of the compound of Formula (IIC) to the iodinating compound is about 1 to 1.1.

In certain embodiments, the halogenation of the compound of Formula (IIC) is carried out in a solvent. Suitable solvents include an ethereal solvent and an aprotic solvent. In certain embodiments as otherwise described herein, the solvent is dimethyl ether, diethyl ether, THF, 2-MeTHF, 1-4-dioxane, 1,2-dimethoxyethane, MTBE, CPME, toluene, or acetonitrile.

In certain embodiments of the process for preparing the compounds of Formula (II) as otherwise described herein, step (d) is carried out in a solution of acetonitrile.

In certain embodiments, the halogenation of the compound of Formula (IIC) is carried out under an inert atmosphere at a temperature in a range of about 40 to about 60° C. Suitable gases for producing an inert atmosphere include nitrogen and argon. Representative temperatures for this reaction are about 45° C., about 50° C., and about 55° C.

In certain embodiments of the process for preparing the compounds of Formula (II) as otherwise described herein, step (d) is carried out under a nitrogen atmosphere at a temperature of about 55° C.

In certain embodiments, the halogenation of the compound of Formula (IIC) is carried out for at least 3 hours. Representative times for completion of this reaction are at least 3.5 hours, at least 4 hours, and at least 4.5 hours.

In certain embodiments of the process for preparing the compounds of Formula (II) as otherwise described herein, step (d) is carried out for at least 4 hours.

In one aspect, the present disclosure provides processes for preparing a compound of Formula (III) (MRTX-1719)

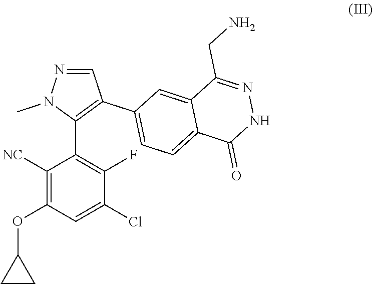 [SMILES: Cn1ncc(-c2ccc3c(=O)[nH]nc(CN)c3c2)c1-c1c(F)c(Cl)cc(OC2CC2)c1C#N]

the process comprising:
(a) treating 4-chloro-2,5-difluorobenzoic acid with a divalent metal base to provide a mixture;
(b) treating the mixture with iodine to provide 4-chloro-3,6-difluoro-2-iodobenzoic acid;
(c) reacting the 4-chloro-3,6-difluoro-2-iodobenzoic acid with an amine to provide 4-chloro-3,6-difluoro-2-iodobenzamide;
(d) dehydrating the 4-chloro-3,6-difluoro-2-iodobenzamide to provide 4-chloro-3,6-difluoro-2-iodobenzonitrile;
(e) coupling the 4-chloro-3,6-difluoro-2-iodobenzonitrile with an organoboron compound to provide 4-chloro-3,6-difluoro-2-(1-methyl-1H-pyrazol-5-yl)benzonitrile;
(f) iodinating the 4-chloro-3,6-difluoro-2-(1-methyl-1H-pyrazol-5-yl)benzonitrile to provide 4-chloro-3,6-difluoro-2-(4-iodo-1-methyl-1H-pyrazol-5-yl)benzonitrile; and
(g) reacting the 4-chloro-3,6-difluoro-2-(4-iodo-1-methyl-1H-pyrazol-5-yl)benzonitrile with cyclopropanol to provide 4-chloro-6-cyclopropoxy-3-fluoro-2-(4-iodo-1-methyl-1H-pyrazol-5-yl)benzonitrile.

In certain embodiments of the process for preparing the compounds of Formula (III) as otherwise described herein, the divalent metal base is TMPMgCl·LiCl, (TMP)₂Mg·2LiCl, or i-PrMgCl·LiCl, and wherein TMP is 2,2,6,6-tetramethylpiperidyl.

In certain embodiments of the process for preparing the compounds of Formula (III) as otherwise described herein, the divalent metal base is (TMP)₂Zn·2LiCl or TMPZnCl·LiCl, and wherein TMP is 2,2,6,6-tetramethylpiperidyl.

In certain embodiments of the process for preparing the compounds of Formula (III) as otherwise described herein, the divalent metal base is dialkylzinc or dialkylzinc halide.

In certain embodiments of the process for preparing the compounds of Formula (III) as otherwise described herein, the divalent metal base is Et₂Zn or EtZnCl.

In certain embodiments of the process for preparing the compounds of Formula (III) as otherwise described herein, step (a) is carried out in a solution of 2-MeTHF under an inert atmosphere at about −20° C.

In certain embodiments of the process for preparing the compounds of Formula (III) as otherwise described herein, step (b) is carried out in a solution of 2-MeTHF under an inert atmosphere at about −15° C.

In certain embodiments of the process for preparing the compounds of Formula (III) as otherwise described herein, in step (b), the iodine is used as a solution of I₂ in 2-MeTHF.

In certain embodiments of the process for preparing the compounds of Formula (III) as otherwise described herein, step (b) further comprises treating the 4-chloro-3,6-difluoro-2-iodobenzoic acid with CDI, followed by treating with the amine in step (c).

In certain embodiments of the process for preparing the compounds of Formula (III) as otherwise described herein, the amine is ammonia or ammonium hydroxide.

In certain embodiments of the process for preparing the compounds of Formula (III) as otherwise described herein, in step (e), the organoboron compound is 1-methyl-5-(4,4,5,5-tetramethyl-1,3,2-dioxaborolan-2-yl)-1H-pyrazole.

In certain embodiments of the process for preparing the compounds of Formula (III) as otherwise described herein, in step (f), the iodinating is carried out with NIS.

In certain embodiments of the process for preparing the compounds of Formula (III) as otherwise described herein, the process further comprises:
(h) coupling the 4-chloro-6-cyclopropoxy-3-fluoro-2-(4-iodo-1-methyl-1H-pyrazol-5-yl)benzonitrile with a protected organoboron phthalazine compound; and
(i) deprotecting the product of step (h).

In certain embodiments, the coupling of the 4-chloro-6-cyclopropoxy-3-fluoro-2-(4-iodo-1-methyl-1H-pyrazol-5-yl)benzonitrile with the protected organoboron phthalazine compound is carried out with a molar ratio of the 4-chloro-6-cyclopropoxy-3-fluoro-2-(4-iodo-1-methyl-1H-pyrazol-5-yl)benzonitrile to the protected organoboron phthalazine compound of about 1.2 to 1 or about 1.5 to 1.

In certain embodiments of the process for preparing the compounds of Formula (III) as otherwise described herein, in step (h), a molar ratio of the 4-chloro-6-cyclopropoxy-3-fluoro-2-(4-iodo-1-methyl-1H-pyrazol-5-yl)benzonitrile to the protected organoboron phthalazine compound is about 1.2 to 1.

In certain embodiments, the coupling of the 4-chloro-6-cyclopropoxy-3-fluoro-2-(4-iodo-1-methyl-1H-pyrazol-5-yl)benzonitrile with the protected organoboron phthalazine compound is carried out in a solvent. Suitable solvents include an ethereal solvent and an aprotic solvent. In certain embodiments as otherwise described herein, the solvent is dimethyl ether, diethyl ether, THF, 2-MeTHF, 1-4-dioxane, 1,2-dimethoxyethane, MTBE, CPME, toluene, or acetonitrile.

In certain embodiments of the process for preparing the compounds of Formula (III) as otherwise described herein, step (h) is carried out in a solution of toluene.

In certain embodiments, the coupling of the 4-chloro-6-cyclopropoxy-3-fluoro-2-(4-iodo-1-methyl-1H-pyrazol-5-yl)benzonitrile with the protected organoboron phthalazine compound is carried out under an inert atmosphere at a temperature in a range of about 50 to about 70° C. Suitable gases for producing an inert atmosphere include nitrogen and argon. Representative temperatures for this reaction are about 55° C., about 60° C., and about 65° C.

In certain embodiments of the process for preparing the compounds of Formula (III) as otherwise described herein, step (h) is carried out at a temperature of about 60° C.

In certain embodiments of the process as otherwise described herein, the protected organoboron phthalazine compound is tert-butyl ((4-oxo-7-(4,4,5,5-tetramethyl-1,3,2-dioxaborolan-2-yl)-3,4-dihydrophthalazin-1-yl)methyl)carbamate.

In certain embodiments of the process as otherwise described herein, the protected organoboron phthalazine compound is prepared by treating tert-butyl ((7-chloro-4-oxo-3,4-dihydrophthalazin-1-yl)methyl)carbamate with 4,4,4′,4′,5,5,5′,5′-octamethyl-2,2′-bi(1,3,2-dioxaborolane) in the presence of tris(dibenzylideneacetone)dipalladium (0).

In certain embodiments, the preparation of the protected organoboron phthalazine compound is carried out with a molar ratio of the tert-butyl ((7-chloro-4-oxo-3,4-dihydrophthalazin-1-yl)methyl)carbamate to the 4,4,4′,4′,5,5,5′,5′-octamethyl-2,2′-bi(1,3,2-dioxaborolane) of about 1 to 1.2, or about 1 to 1.5, or about 1 to 1.8, or about 1 to 2.

In certain embodiments of the process for preparing the protected organoboron phthalazine compound as otherwise described herein, a molar ratio of the tert-butyl ((7-chloro-4-oxo-3,4-dihydrophthalazin-1-yl)methyl)carbamate to the 4,4,4′,4′,5,5,5′,5′-octamethyl-2,2′-bi(1,3,2-dioxaborolane) is about 1 to 1.5.

In certain embodiments, the preparation of the protected organoboron phthalazine compound is carried out with a molar ratio of the tert-butyl ((7-chloro-4-oxo-3,4-dihydrophthalazin-1-yl)methyl)carbamate to the tris(dibenzylideneacetone)dipalladium (0) of about 1 to 0.10, or about 1 to 0.15, or about 1 to 0.2.

In certain embodiments of the process for preparing the protected organoboron phthalazine compound as otherwise described herein, a molar ratio of the tert-butyl ((7-chloro-4-oxo-3,4-dihydrophthalazin-1-yl)methyl)carbamate to the tris(dibenzylideneacetone)dipalladium (0) is about 1 to 0.10.

In certain embodiments, the preparation of the protected organoboron phthalazine compound is carried out in the presence of a base. Suitable bases include sodium acetate, potassium acetate, and magnesium acetate.

In certain embodiments of the process for preparing the compounds of Formula (III) as otherwise described herein, in step (h), the preparation of the protected organoboron phthalazine compound is carried out in the presence of potassium acetate.

In certain embodiments, the preparation of the protected organoboron phthalazine compound is carried out in a solvent. Suitable solvents include an ethereal solvent and an aprotic solvent. In certain embodiments as otherwise described herein, the solvent is dimethyl ether, diethyl ether, THF, 2-MeTHF, 1-4-dioxane, 1,2-dimethoxyethane, MTBE, CPME, toluene, or acetonitrile.

In certain embodiments of the process for preparing the compounds of Formula (III) as otherwise described herein, in step (h), the preparation of the protected organoboron phthalazine compound is carried out in a solution of 1,4-dioxane.

In certain embodiments, the preparation of the protected organoboron phthalazine compound is carried out under an inert atmosphere at a temperature in a range of about 80 to about 120° C. Suitable gases for producing an inert atmosphere include nitrogen and argon. Representative temperatures for this reaction are about 90° C., about 100° C., and about 110° C.

In certain embodiments of the process for preparing the compounds of Formula (III) as otherwise described herein, in step (h), the preparation of the protected organoboron phthalazine compound is carried out under a nitrogen atmosphere at about 100° C.

In certain embodiments, the deprotection of the product of step (h) is carried out under an acidic condition. Suitable acids for producing the acidic condition include HCl and trifluoroacetic acid (TFA).

In certain embodiments of the process for preparing the compounds of Formula (III) as otherwise described herein, in step (h), the product of step (h) is deprotected using HCl or TFA.

In certain embodiments, the deprotection of the product of step (h) is carried out at a temperature in a range of about 10 to about 40° C. Representative temperatures for this reaction are about 15° C., about 20° C., about 25° C., about 30° C., and about 35° C. In certain embodiments of the process for preparing the compounds of Formula (III) as otherwise described herein, in step (h), the product of step (h) is deprotected at a temperature of about 30° C.

### Compounds and Compositions

In one aspect, the present disclosure provides compounds of Formula (IV)

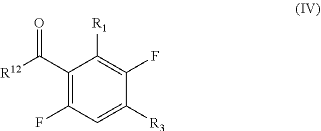

wherein:
   R¹ is iodo, an aryl group, or a heteroaryl group;
   R³ is bromo or chloro; and
   R¹² is-OR¹³ or —NR¹⁴R¹⁵, wherein each of R¹³, R¹⁴, and R¹⁵ is independently hydrogen, C₁-C₆ alkyl, or C₃-C₆ cycloalkyl.

In certain embodiments of the compounds of Formula (IV) as otherwise described herein, R¹ is iodo.

In certain embodiments of the compounds of Formula (IV) as otherwise described herein, R¹ is an aryl group.

In certain embodiments of the compounds of Formula (IV) as otherwise described herein, R¹ is a C₆-C₁₀ aryl group optionally substituted with one or more of C₁-C₆ alkyl, halo C₁-C₆ alkyl, C₃-C₆ cycloalkyl, or C₁-C₆ alkoxy.

In certain embodiments of the compounds of Formula (IV) as otherwise described herein, the aryl group is phenyl, naphthyl, anthracenyl, fluorenyl, benzyl, phenethyl, or naphthylmethyl. In certain embodiments of the compounds of Formula (IV) as otherwise described herein, R¹ is a heteroaryl group.

In certain embodiments of the compounds of Formula (IV) as otherwise described herein, R¹ is a 5- or 6-member heteroaryl group optionally substituted with one or more of C₁-C₆ alkyl, halo C₁-C₆ alkyl, C₃-C₆ cycloalkyl, or C₁-C₆ alkoxy.

In certain embodiments of the compounds of Formula (IV) as otherwise described herein, the heteroaryl group is furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, or triazinyl.

In certain embodiments of the compounds of Formula (IV) as otherwise described herein, R³ is chloro.

In certain embodiments of the compounds of Formula (IV) as otherwise described herein, R³ is bromo.

In certain embodiments of the compounds of Formula (IV) as otherwise described herein, R¹² is —OH.

In certain embodiments of the compounds of Formula (IV) as otherwise described herein, R¹² is —NH₂.

In another aspect, the present disclosure provides compositions comprising a mixture of 4-chloro-2,5-difluorobenzoic acid and 4-chloro-3,6-difluoro-2-iodobenzoic acid.

In certain embodiments of the compositions as otherwise described herein, the mixture is substantially free of 4-chloro-2,5-difluoro-3-iodobenzoic acid.

In certain embodiments of the compositions as otherwise described herein, the mixture comprises greater than about 90% (w/w) of 4-chloro-3,6-difluoro-2-iodobenzoic acid and less about 10% of 4-chloro-2,5-difluoro-3-iodobenzoic acid based on the combined weight of 4-chloro-3,6-difluoro-2-iodobenzoic acid and 4-chloro-2,5-difluoro-3-iodobenzoic acid. In certain embodiments of the compositions as otherwise described herein, the mixture comprises 4-chloro-3,6-difluoro-2-iodobenzoic acid and 4-chloro-2,5-difluoro-3-iodobenzoic acid in a ratio of 99:1.

In certain embodiments of the compositions as otherwise described herein, the composition further comprises a divalent base.

In certain embodiments of the compositions as otherwise described herein, the divalent base is TMPMgCl·LiCl, (TMP)₂Mg·2LiCl, or i-PrMgCl·LiCl, and wherein TMP is 2,2,6,6-tetramethylpiperidyl.

In certain embodiments of the compositions as otherwise described herein, the divalent metal base is (TMP)₂Zn·2LiCl or TMPZnCl·LiCl, and wherein TMP is 2,2,6,6-tetramethylpiperidyl.

In certain embodiments of the compositions as otherwise described herein, the divalent metal base is dialkylzinc or dialkylzinc halide.

In certain embodiments of the compositions as otherwise described herein, the divalent metal base is Et₂Zn or EtZnCl.

In certain embodiments of the compositions as otherwise described herein, the composition further comprises iodine.

### Methods for Separation of Enatiomers

The compound of Formula (III) (MRTX-1719) as prepared by the processes described herein is axially chiral and comprises a mixture of M- and P-enantiomers thereof.

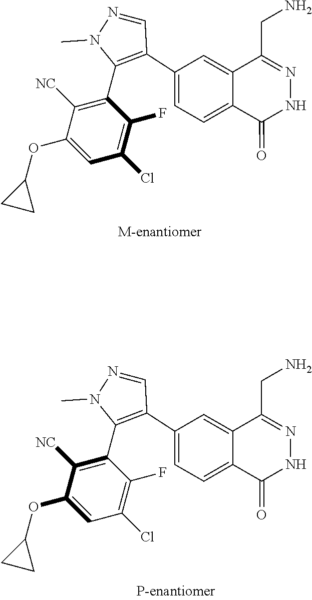

However, only the M-enantiomer is pharmacologically active. Methods for separating the mixture of the M- and P-enantiomers is described in International Application Publication No. 2023/059798, published Apr. 13, 2023, the disclosure of which is hereby incorporated by reference in its entirety.

Briefly, in one aspect, the separation methods comprise:
(a) treating the mixture with N-Boc-D-phenylalanine to form a mixture of M- and P-enantiomer N-Boc-D-phenylalanine salts;
(b) filtering the mixture of M- and P-enantiomer N-Boc-D-phenylalanine salts to obtain a solid phase enriched with the M-enantiomer N-Boc-D-phenylalanine salt; and
(c) reacting the solid phase with a base to obtain a solid enriched with the M-enantiomer of the compound of Formula (IV) as a free base.

In one aspect, the separation methods comprise:
(a) treating the mixture with N-Boc-L-phenylalanine to form a mixture of M- and P-enantiomer N-Boc-L-phenylalanine salts;
(b) filtering the mixture of M- and P-enantiomer N-Boc-L-phenylalanine salts to obtain a liquid phase enriched with the M-enantiomer N-Boc-L-phenylalanine salt; and
(c) reacting the liquid phase with a base to obtain a solid enriched with the M-enantiomer of the compound of Formula (III) as a free base.

In one aspect, the methods comprise:
(a) protecting the M- and P-enantiomers of the compound of Formula (III) with a protecting group;
(b) chromatographically separating the protected M-enantiomer of the compound of Formula (III) from the protected P-enantiomer of the compound of Formula (III); and
(c) deprotecting the protected M-enantiomer of the compound of Formula (III) to provide the M-enantiomer of the compound of Formula (III) as a free base.

Other aspects and embodiments of the methods for separation are evident in view of the detailed description provided herein.

### General Reaction Schemes and Examples

The compounds of the present invention may be prepared using commercially available reagents and conventional methods well known to those skilled in the art, for example, in General Reaction Schemes and Examples as described herein. One of ordinary skill in the art can adapt the reaction sequences of schemes as provided herein to fit the desired target molecule. Of course, in certain situations one of skill in the art will use different reagents to affect one or more of the individual steps or to use protected versions of certain of the substituents. Additionally, one of ordinary skill in the art would recognize that compounds of the disclosure can be synthesized using different routes altogether. For example, one of ordinary skill in the art may adapt the procedures described herein and/or other procedures familiar to the one of ordinary skill in the art to make the compounds described herein.

Many general references providing commonly known chemical synthetic schemes and conditions useful for synthesizing the disclosed compounds are available. For example, the compounds of the present invention can be prepared using the reagents and methods as described in International Application Publication No. 2021/050915, published Mar. 18, 2021, and International Application Publication No. 2023/059798, published Apr. 13, 2023, the disclosure of each of which is hereby incorporated by its entirety.

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed. E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/1, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

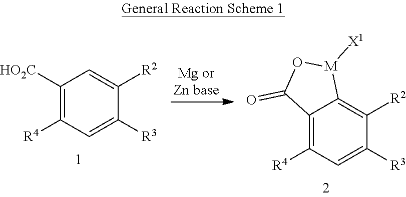

wherein M represents Mg or Zn of the divalent metal base, and X¹ represents a counterion of the divalent metal base.

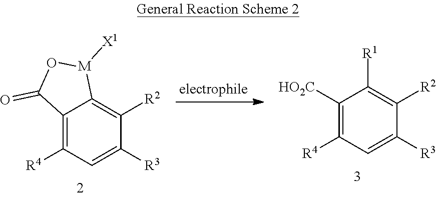

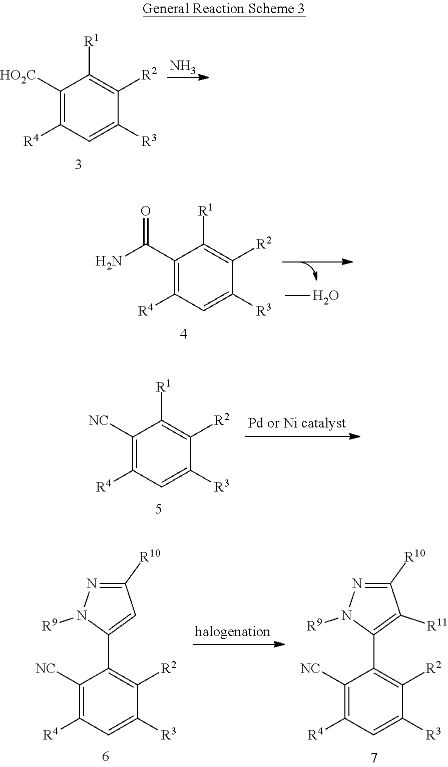

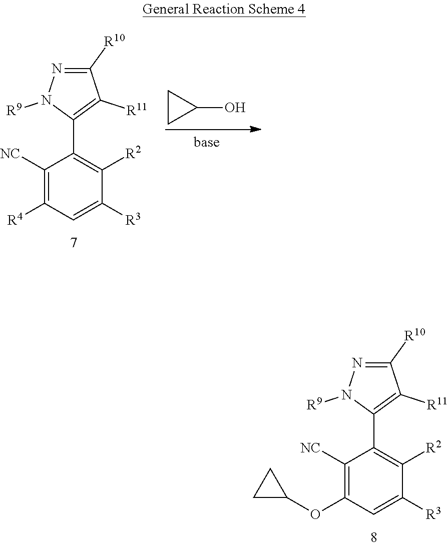

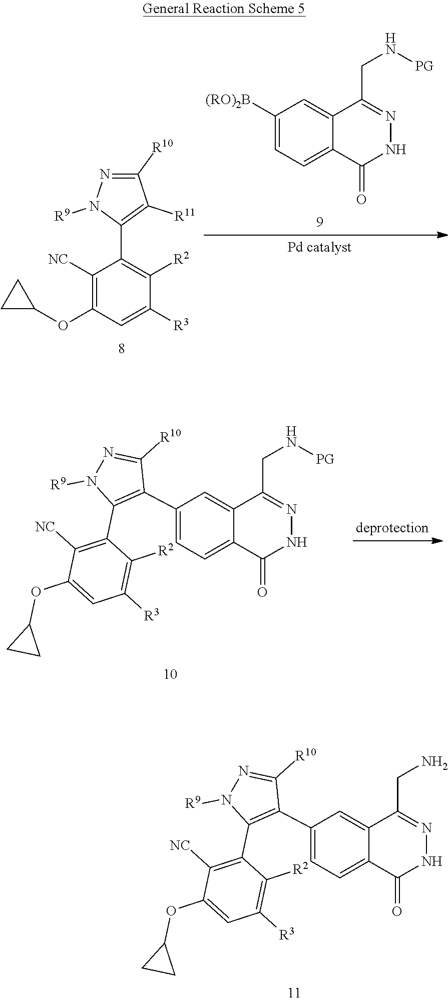

wherein PG presents a protecting group, and R is hydrogen or C₁-C₆ alkyl.

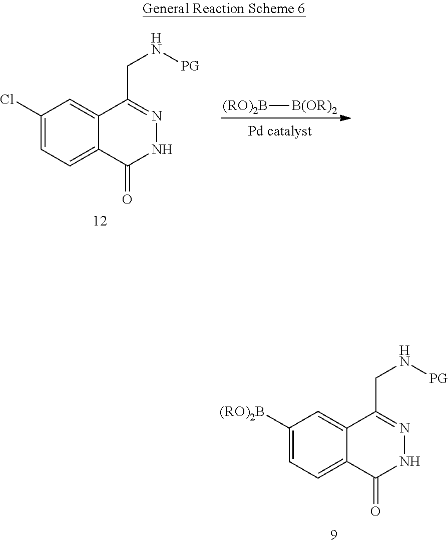

### Example 1

### Synthesis of 4-chloro-3,6-difluoro-2-iodobenzoic acid

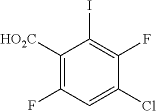 [SMILES: O=C(O)c1c(F)cc(Cl)c(F)c1I]

4-Chloro-2,5-difluorobenzoic acid (1.5 kg, 7.8 mol) is dissolved in 2-MeTHF (6.0 L), and the 10 solution is degassed with nitrogen and stirred at room temperature. The solution is then cooled to about −25° C. under nitrogen, and 1.0 M TMPMgCl·LiCl solution in 2-MeTHF (17.2 L, 2.2 equiv.; TMP=2,2,6,6-tetramethylpiperidyl) is added to the solution while reaction temperature maintains at about −20° C. The resulting reaction mixture is stirred at about −20° C. for about 3 h to provide the first reaction mixture. In another vessel, a solution of iodine (2.5 kg, 9.8 mol) in 2-MeTHF (6.0 L) is prepared, and the solution is degassed with nitrogen and stirred at room temperature to provide a second reaction mixture. The second reaction mixture is then cooled to about −35° C. To the second reaction mixture is added the first reaction mixture at a temperature of no more than −25° C., and the resulting mixture is stirred at about −25° C. for about 1 h. To the resulting mixture is slowly added saturated aqueous NH₄Cl (4.5 L) at a temperature of no more than 10° C., followed by slowly adding saturated aqueous NaHSO₃ (9.8 L) at a temperature of no more than 10° C. The resulting mixture is concentrated by distillation under reduced pressure at a temperature of no more than 45° C., and to the residue is added methyl tert-butyl ether (15 L; MTBE). The resulting mixture is stirred at about 20° C. for about 0.5 h. To the resulting mixture is slowly added 6N HCl solution until the pH of the aqueous layer is between 1 and 2, followed by filtration. The waste filter cake is rinsed with MTBE (2×3 L), and all the MTBE filtrates are combined. The resulting organic layer is washed with water (7.5 L) and then subject to solvent exchange to THF (target: <5 L) through distillation under reduced pressure at a temperature of no more than 45° C. to afford a colorless or brown solution of 4-chloro-3,6-difluoro-2-iodobenzoic acid in THF (5.35 kg, 7.0 mol, 90% assay yield), which can be used in subsequent synthesis without further purification.

### Example 2

### Synthesis of 4-chloro-3,6-difluoro-2-(4-iodo-1-methyl-1H-pyrazol-5-yl)benzonitrile

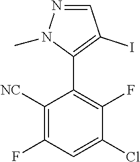 [SMILES: Cn1ncc(I)c1-c1c(F)c(Cl)cc(F)c1C#N]

### Step 1: Synthesis of 4-chloro-3,6-difluoro-2-iodobenzamide

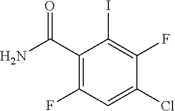 [SMILES: NC(=O)c1c(F)cc(Cl)c(F)c1I]

To a solution of 4-chloro-3,6-difluoro-2-iodobenzoic acid in THF (5.35 kg, 7.0 mol) is added carbonyl diimidazole (2.3 kg, 14 mol, 2.0 equiv.; CDI) portionwise at a temperature of no more than 25° C., and the reaction mixture is degassed with nitrogen and stirred at about 20° C. for about 0.5 h. The reaction mixture is then stirred at about 55° C. for at least 2 h. To the reaction mixture is added ˜28 wt % aqueous ammonium hydroxide (6.7 L) at a temperature of no more than 55° C., and the resulting reaction mixture is stirred at about 50° C. for at least 1 h. The resulting mixture is concentrated by distillation under reduced pressure at a temperature of no more than 45° C., and to the residue is added water (11 L). The residue is stirred at about 20° C. for at least 1 h, followed by filtration. The filtered residue are reslurried in water (6.7 L) and then heptane (6.7 L), and the resulting residue is vacuum-dried at a temperature of no more than 55° C. to afford 4-chloro-3,6-difluoro-2-iodobenzamide as a solid (2.1 kg, 6.6 mol, 95% yield).

### Step 2: Synthesis of 4-chloro-3,6-difluoro-2-iodobenzonitrile

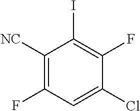 [SMILES: N#Cc1c(F)cc(Cl)c(F)c1I]

4-Chloro-3,6-difluoro-2-iodobenzamide (2.1 kg, 6.6 mol) is dissolved in THF (10.5 L) at room temperature, and then degassed with nitrogen. The solution is then cooled to about 0° C. To the solution is added triethylamine (2.4 L, 17 mol, 2.6 equiv.; Et₃N) dropwise at a temperature of no more than 10° C., followed by adding trifluoroacetic anhydride (1.2 L, 8.6 mol, 1.3 equiv.; TFAA) at a temperature of no more than 10° C. The resulting mixture is stirred at a temperature of no more than 10° C. for about 0.5 h and then stirred at a temperature of no more than 25° C. for at least 2 h. The resulting mixture is then cooled to about 0° C. To the resulting mixture is added water (2.1 L) dropwise at a temperature of no more than 10° C., and the resulting mixture is stirred at a temperature of no more than 10° C. for about 0.5 h. The resulting mixture is then concentrated by distillation under reduced pressure at a temperature of no more than 45° C., and to the residue is added 5% w/w aqueous K₂CO₃ (8.4 L). The resulting residue is stirred at a temperature of no more than 25° C. for about 0.5 h, followed by filtration. The filtered residue is washed with water (4.2 L) and vacuum-dried at a temperature of no more than 60° C. to afford 4-chloro-3,6-difluoro-2-iodobenzonitrile as a solid (1.88 kg, 6.27 mol, 95% yield).

### Step 3: Synthesis of 4-chloro-3,6-difluoro-2-(1-methyl-1H-pyrazol-5-yl)benzonitrile

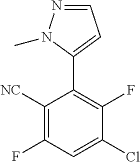 [SMILES: Cn1nccc1-c1c(F)c(Cl)cc(F)c1C#N]

A mixture of toluene (19 L), 4-chloro-3,6-difluoro-2-iodobenzonitrile (1.88 kg, 6.27 mol), 1-methyl-5-(4,4,5,5-tetramethyl-1,3,2-dioxaborolan-2-yl)-1H-pyrazole (1.50 kg, 7.2 mol, 1.15 equiv.), and K₃PO₄ (2.8 kg, 13 mol, 2.0 equiv.) is degassed with nitrogen and stirred at room temperature with subsurface nitrogen sparging for about 0.5 h. To the reaction mixture is added [P(t-Bu)₂(n-Bu)]—Pd-G3 (3.0 mol %, 0.19 mol), and the reaction mixture is stirred at room temperature with subsurface nitrogen sparging for about 0.5 h. The reaction mixture is then heated to about 70° C., and to the reaction mixture is added degassed water (0.94 L). The reaction mixture is then stirred at about 75° C. for about 20 h. The reaction mixture is then cooled to 20° C., and to the reaction mixture is then added water (19 L), followed by filtration. The resulting organic layer is concentrated by distillation under reduced pressure at a temperature of no more than 55° C., and to the residue is added heptane (9.4 L) at about 55° C. The resulting residue is cooled to about 20° C. and stirred for at least 5 h, followed by filtration. The filtered residue is washed with heptane (2×2 L) and vacuum-dried at a temperature of no more than 45° C. to afford 4-chloro-3,6-difluoro-2-(1-methyl-1H-pyrazol-5-yl)benzonitrile as a solid (1.11 kg g, 4.39 mol, 70% yield).

### Step 4: Synthesis of 4-chloro-3,6-difluoro-2-(4-iodo-1-methyl-1H-pyrazol-5-yl)benzonitrile

4-Chloro-3,6-difluoro-2-(1-methyl-1H-pyrazol-5-yl)benzonitrile (1.11 kg, 4.39 mol) is solubilized in acetonitrile (6.6 L) at room temperature, and the solution is degassed with nitrogen. To the solution is added N-iodosuccinimide (1.09 kg, 4.8 mol, 1.1 equiv.; NIS) at a temperature of about 20° C., followed by adding trifluoroacetic acid (0.17 L, 2.2 mol) dropwise at a temperature of no more than 25° C. The reaction mixture is then heated to about 55° C. and stirred for at least 4 h. The reaction mixture is concentrated by distillation under reduced pressure at a temperature of no more than 45° C., and to the residue is added MTBE (11 L) at about 20° C. To the resulting residue is added 15% w/w aqueous Na₂SO₃ (3.3 L), and the resulting residue is stirred at about 20° C. for at least 0.5 h. The resulting organic layer is washed with 5% w/w aqueous NaHCO₃ (6.1 L) and brine (6.6 L) and concentrated by distillation under reduced pressure at a temperature of no more than 45° C., and to the residue is added heptane (11 L). The resulting residue is cooled to 20° C. and stirred for at least 5 h, followed by filtration. The filtered residue is washed with heptane (3.3 L) and vacuum-dried at a temperature of no more than 45° C. to afford 4-chloro-3,6-difluoro-2-(4-iodo-1-methyl-1H-pyrazol-5-yl)benzonitrile as a solid (1.45 kg, 3.82 mol, 87% yield).

### Example 3

### Synthesis of 4-chloro-6-cyclopropoxy-3-fluoro-2-(4-iodo-1-methyl-1H-pyrazol-5-yl)benzonitrile

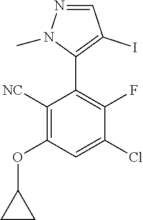 [SMILES: Cn1ncc(I)c1-c1c(F)c(Cl)cc(OC2CC2)c1C#N]

A mixture of dimethylformamide (8.7 L; DMF), 4-chloro-3,6-difluoro-2-(4-iodo-1-methyl-1H-pyrazol-5-yl)benzonitrile (1.45 kg, 3.82 mol), cyclopropanol (233 g, 4.01 mol, 1.05 equiv.; cPrOH), and K₃PO₄ (2.43 kg, 11.5 mol, 3.0 equiv.) is degassed with nitrogen at room temperature. To the reaction mixture is added water (0.22 L) at about 20° C. The reaction mixture is then heated to about 35° C. and stirred for at least 15 h. Thereafter, the reaction mixture is heated to about 45° C. and stirred for at least 2 h. The reaction mixture is then cooled to about 0° C. To the reaction mixture is slowly added water (17 L) at about 0° C., and the resulting mixture is stirred for at least 3 h, followed by filtration. The resulting residue is a crude product of 4-chloro-6-cyclopropoxy-3-fluoro-2-(4-iodo-1-methyl-1H-pyrazol-5-yl)benzonitrile. The crude product is then dissolved in acetone (8.7 L). To the resulting mixture is added water (8.7 L) at about 20° C., and the resulting mixture is stirred for at least 3 h, followed by filtration. The filtered residue is vacuum-dried at a temperature of no more than 60° C. to afford a purified product of 4-chloro-6-cyclopropoxy-3-fluoro-2-(4-iodo-1-methyl-1H-pyrazol-5-yl)benzonitrile as a solid (1.28 kg, 3.06 mol, 80% yield).

### Example 4

### Synthesis of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile

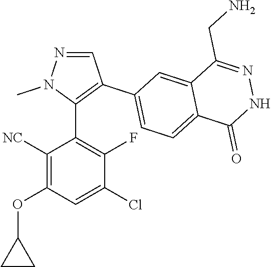 [SMILES: Cn1ncc(-c2ccc3c(=O)[nH]nc(CN)c3c2)c1-c1c(F)c(Cl)cc(OC2CC2)c1C#N]

### Step 1: Synthesis of tert-butyl ((7-(5-(3-chloro-6-cyano-5-cyclopropoxy-2-fluorophenyl)-1-methyl-1H-pyrazol-4-yl)-4-oxo-3,4-dihydrophthalazin-1-yl)methyl)carbamate

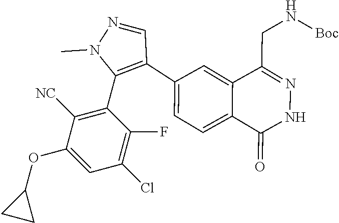 [SMILES: Cn1ncc(-c2ccc3c(=O)[nH]nc(CNC(=O)OC(C)(C)C)c3c2)c1-c1c(F)c(Cl)cc(OC2CC2)c1C#N]

A 1000 L reactor is charged with 4-chloro-6-cyclopropoxy-3-fluoro-2-(4-iodo-1-methyl-1H-pyrazol-5-yl)benzonitrile (18.1 kg, 43.3 mol, 1.00 equiv.), tert-butyl ((4-oxo-7-(4,4,5,5-tetramethyl-1,3,2-dioxaborolan-2-yl)-3,4-dihydrophthalazin-1-yl)methyl)carbamate (19.5 kg, 48.5 mol, 1.12 equiv.), toluene (12 vol; vol=L/kg), water (4 vol), and Cs₂CO₃ (42.4 kg, 130 mol, 3.00 equiv.). The mixture is stirred for about 15 minutes at 15-25° C. and then vacuum-degassed and backfilled with nitrogen (repeat three times). Ad₂nBuP-Pd-G3 (946 g, 1.30 mol, 0.03 equiv.) is added, and the mixture is vacuum degassed and backfilled with nitrogen (repeat three times). The contents of the reactor are heated to 55-60° C., and agitation continues at the temperature range until Suzuki coupling is complete (<5.0% of 4-chloro-6-cyclopropoxy-3-fluoro-2-(4-iodo-1-methyl-1H-pyrazol-5-yl)benzonitrile remaining by LC). The batch is cooled to 45-50° C. An aqueous cysteine solution (20 w/w %) and 2-MeTHF (12 vol) are added to the reactor, and the biphasic mixture is stirred for about 6 to 8 hours at 45-50° C. The aqueous phase is discarded, and the organic phase is washed with aqueous 17% NaCl (2×5 vol). Anhydrous MgSO₄ (18 kg, 100 w/w %) and activated charcoal (3.6 kg, 20 w/w %) are added to the crude organic layer and agitated at 45-50° C. for about 4 to 6 hours. The inorganic solids are filtered, and the waste cake is washed with 2-MeTHF (vol mL). The combined organic filtrate is concentrated to 2 to 4 mL under reduced pressure at 30-60° C. Put-and-take distillation with MeOH (3 vol) under reduced pressure at 30-60° C. is performed repeatedly to reduce toluene content (<5.0%). The batch (about 3 vol) is diluted with MeOH (7 vol) and stirred at 45-50° C. for about 6 hours. The batch is cooled to 20-25° C. and aged with agitation for about 12 hours to reduce the supernatant concentration of tert-butyl ((7-(5-(3-chloro-6-cyano-5-cyclopropoxy-2-fluorophenyl)-1-methyl-1H-pyrazol-4-yl)-4-oxo-3,4-dihydrophthalazin-1-yl)methyl)carbamate (<5.0%). The solids are collected by filtration and washed with MeOH (2 vol). If the purity of the wet cake is insufficient (any single impurity >1.5% by LC), additional slurry in MeOH (8 vol) is performed. The wet cake is dried at 60-65° C. for about 24 hours (LOD <10.0%) to afford tert-butyl ((7-(5-(3-chloro-6-cyano-5-cyclopropoxy-2-fluorophenyl)-1-methyl-1H-pyrazol-4-yl)-4-oxo-3,4-dihydrophthalazin-1-yl)methyl)carbamate as an off-white solid (18.3 kg, 99.0% LC purity, 91.5 wt % assay by QNMR, 74.7% isolated yield, 22 ppm Pd by ICP-MS).

### Step 2: Synthesis of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (MRTX-1719)

A 1000 L reactor is charged with tert-butyl ((7-(5-(3-chloro-6-cyano-5-cyclopropoxy-2-fluorophenyl)-1-methyl-1H-pyrazol-4-yl)-4-oxo-3,4-dihydrophthalazin-1-yl)methyl)carbamate (18.2 kg, 32.2 mol, 1.00 equiv.) and EtOAc (15 vol; vol=L/kg) at 10-20° C. 4 M HCl in EtOAc (5 vol) is added slowly to the mixture while the internal temperature maintains at 10-20° C. The mixture is stirred at 15-25° C. for about 18 hours to effect complete deprotection of tert-butyl ((7-(5-(3-chloro-6-cyano-5-cyclopropoxy-2-fluorophenyl)-1-methyl-1H-pyrazol-4-yl)-4-oxo-3,4-dihydrophthalazin-1-yl)methyl)carbamate (<0.3% remaining by LC). The resulting slurry is filtered, and the filter cake is washed with EtOAc (10 vol) at 20-25° C. The wet cake is vacuum-dried at 40-45° C. for about 6 to 8 hours (LOD <10.0%) to afford 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile·HCl. A 500 L reactor is charged with the 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile·HCl and MeOH (10 vol) at 10-15° C. 7 M NH₃ in MeOH (1 vol) is added slowly while the internal temperature maintains at 10-15° C. The resulting slurry is agitated at 10-15° C. for about 12 hours to reduce the supernatant content of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (<1.0% by LC). The slurry is centrifuged at 10-15° C., and the wet cake is slurried in water (2×10 vol) to remove residual NH₄Cl (<0.05 wt % by IC) to afford 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile as an off-white solid (24.1 kg, 99.3% LC purity, 56.1 wt % assay by QNMR, 89.0% isolated yield, 6 ppm Pd by ICP-MS).

### Example 5

### Synthesis of tert-butyl ((4-oxo-7-(4,4,5,5-tetramethyl-1,3,2-dioxaborolan-2-yl)-3,4-dihydrophthalazin-1-yl)methyl)carbamate

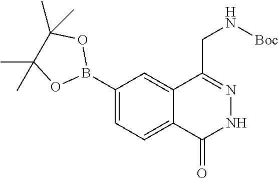 [SMILES: CC(C)(C)OC(=O)NCc1n[nH]c(=O)c2ccc(B3OC(C)(C)C(C)(C)O3)cc12]

To a 40 mL vial containing a magnetic stirring bar are added tert-butyl ((7-chloro-4-oxo-3,4-dihydrophthalazin-1-yl)methyl)carbamate (1.0 g, 3.2 mmol, 1.0 equiv.), 4,4,4′,4′,5,5,5′,5′-octamethyl-2,2′-bi(1,3,2-dioxaborolane) (1.2 g, 4.8 mmol, 1.5 equiv.), potassium acetate (951 mg, 9.7 mmol, 3.0 equiv.), XPhos (3.8 mg, 8.1 μmol, 0.0025 equiv.) and 1,4-dioxane (10 mL). The mixture is degassed by bubbling nitrogen to the solution for about 2 minutes. Tris(dibenzylideneacetone)dipalladium (0) (3.7 mg, 4.0 μmol, 0.00125 equiv.) is added against nitrogen flow, and the reaction is sealed with electric tape. The reaction mixture is stirred at 100° C. for about 2 hours. The reaction is then cooled to 50° C., and water (15 mL) is slowly added. The mixture is then allowed to cool to 20° C., and stirring continues for about 1 hour. The resulting crystalline solid is isolated by filtration and rinsed with a 1,4-dioxane/water solution (40/60 v/v, 3.0 mL) to afford tert-butyl ((4-oxo-7-(4,4,5,5-tetramethyl-1,3,2-dioxaborolan-2-yl)-3,4-dihydrophthalazin-1-yl)methyl)carbamate as a white crystalline solid (1.18 g, 2.9 mmol, 91% yield).

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A process for preparing a compound of Formula (I) 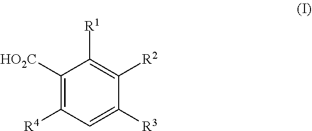
wherein:
R¹ is halogen, an aryl group, or a heteroaryl group;
each of R² and R³ is independently halogen; and
R⁴ is halogen, C₁-C₆ alkyl, C₃-C₆ cycloalkyl, halo C₁-C₆ alkyl, or OR⁵, wherein R⁵ is C₁-C₆ alkyl, C₃-C₆ cycloalkyl, or halo C₁-C₆ alkyl,
the process comprising treating a compound of Formula (IA) 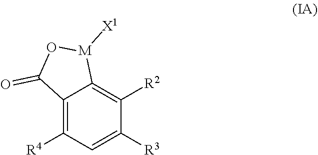
with an electrophile which is I₂, Br₂, Cl₂, an aryl compound, or a heteroaryl compound,
wherein:
M represents a divalent metal of a divalent metal base; and
X¹ represents a counterion of the divalent metal base.

2. The process of claim 1, wherein R¹ is halogen.

3. The process of claim 2, wherein R¹ is iodo.

4. The process of claim 1, wherein R¹ is an aryl group.

5. The process of claim 4, wherein R¹ is a C₆-C₁₀ aryl group optionally substituted with one or more of C₁-C₆ alkyl, halo C₁-C₆ alkyl, C₃-C₆ cycloalkyl, or C₁-C₆ alkoxy.

6. The process of claim 4 or claim 5, wherein the aryl group is phenyl, naphthyl, anthracenyl, fluorenyl, benzyl, phenethyl, or naphthylmethyl.

7. The process of claim 1, wherein R¹ is a heteroaryl group.

8. The process of claim 7, wherein R¹ is a 5- or 6-member heteroaryl group optionally substituted with one or more of C₁-C₆ alkyl, halo C₁-C₆ alkyl, C₃-C₆ cycloalkyl, or C₁-C₆ alkoxy.

9. The process of claim 7 or claim 8, wherein the heteroaryl group is furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, or triazinyl.

10. The process of claim 1, wherein the electrophile is a heteroaryl compound of Formula (IB) 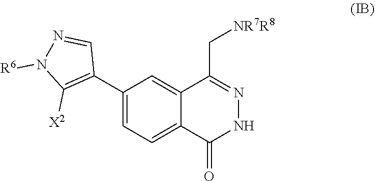
wherein:
X² is I, Br, or C₁;
R⁶ is hydrogen, C₁-C₆ alkyl, C₃-C₆ cycloalkyl, or halo C₁-C₆ alkyl; and
each of R⁷ and R⁸ is independently hydrogen or a protecting group, or R⁷ and R⁸ together with the N to which they bind form a ring structure.

11. The process of claim 10, wherein R⁶ is methyl.

12. The process of claim 10 or claim 11, wherein each of R⁷ and R⁸ is independently hydrogen.

13. The process of claim 10 or claim 11, wherein one of R⁷ and R⁸ is hydrogen, and the other is a protecting group.

14. The process of claim 10 or claim 13, wherein the protecting group is a tert-butyloxycarbonyl (Boc) group.

15. The process of claim 10, wherein the ring structure is a phthalimide group.

16. The process of any of claims 1-15, wherein R² is fluoro.

17. The process of any of claims 1-16, wherein R³ is chloro.

18. The process of any of claims 1-16, wherein R³ is bromo.

19. The process of any of claims 1-18, wherein R⁴ is fluoro.

20. The process of any of claims 1-18, wherein R⁴ is OR⁵, and wherein R⁵ is cyclopropyl.

21. The process of any of claims 1-20, wherein the compound of Formula (IA) is in an ethereal solvent.

22. The process of any of claims 1-21, wherein the process is carried out in an ethereal solvent.

23. The process of claim 21 or claim 22, wherein the ethereal solvent is tetrahydrofuran (THF), 2-methyltetrahydrofuran (2-MeTHF), 1-4-dioxane, 1,2-dimethoxyethane, methyl tert-butyl ether (MTBE), or cyclopentyl methyl ether (CPME).

24. The process of any of claims 1-23, wherein the process is carried out under an inert atmosphere at a temperature in a range of −10 to −30° C.

25. The process of any of claims 1-24, wherein the compound of Formula (IA) is prepared by treating a compound of Formula (IC) 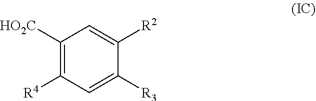
with the divalent metal base M-X¹.

26. The process of claim 25, wherein the treatment of the compound of Formula (IC) is carried out in an aprotic solvent.

27. The process of claim 25 or claim 26, wherein the treatment of the compound of Formula (IC) is carried out under an inert atmosphere at a temperature of −30° C. or higher.

28. The process of any of claims 1-27, wherein the divalent metal base is a magnesium base.

29. The process of claim 28, wherein the magnesium base is an alkyl Grignard reagent.

30. The process of claim 28, wherein the magnesium base is TMPMgCl·LiCl, (TMP)₂Mg·2LiCl, or i-PrMgCl·LiCl, and wherein TMP is 2,2,6,6-tetramethylpiperidyl.

31. The process of any of claims 28-30, wherein the magnesium base is in a solution of THF or 2-MeTHF.

32. The process of any of claims 1-27, wherein the divalent metal base is a zinc base.

33. The process of claim 32, wherein the zinc base is (TMP)₂Zn·2LiCl or TMPZnCl·LiCl, and wherein TMP is 2,2,6,6-tetramethylpiperidyl.

34. The process of claim 32, wherein the zinc base is dialkylzinc or dialkylzinc halide.

35. The process of claim 34, wherein the zinc base is Et₂Zn or EtZnCl.

36. The process of any of claims 1-35, wherein the compound of Formula (I) is 4-chloro-3,6-difluoro-2-iodobenzoic acid, which is prepared by:
(a) treating 4-chloro-2,5-difluorobenzoic acid with the divalent metal base to provide a mixture; and
(b) treating the mixture with iodine to provide 4-chloro-3,6-difluoro-2-iodobenzoic acid.

37. A process for preparing a compound of Formula (II) 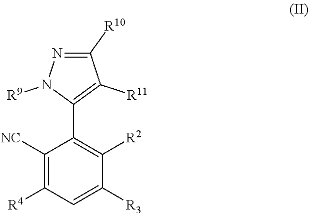
wherein:
each of R², R³, and R¹¹ is independently halogen;
R⁴ is halogen, C₁-C₆ alkyl, C₃-C₆ cycloalkyl, halo C₁-C₆ alkyl, or OR⁵, wherein R⁵ is C₁-C₆ alkyl, C₃-C₆ cycloalkyl, or halo C₁-C₆ alkyl;
R⁹ is C₁-C₆ alkyl, C₃-C₆ cycloalkyl, or halo C₁-C₆ alkyl; and
R¹⁰ is hydrogen,
the process comprising:
(a) reacting a compound of Formula (I) 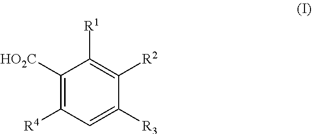
with an amine to provide a compound of Formula (IIA) 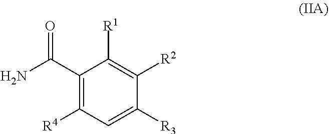
wherein R¹ is halogen, an aryl group, or a heteroaryl group;
(b) dehydrating the compound of Formula (IIA) to provide a compound of Formula (IIB) 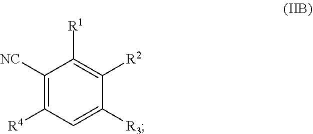
(c) coupling the compound of Formula (IIB) with an organoboron compound or an organozinc compound to provide a compound of Formula (IIC) 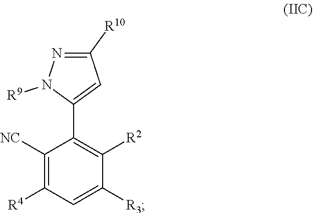
and
(d) halogenating the compound of Formula (IIC) to provide the compound of Formula (II).

38. The process of claim 37, wherein R² is fluoro.

39. The process of claim 37 or claim 38, wherein R³ is chloro.

40. The process of claim 37 or claim 38, wherein R³ is bromo.

41. The process of any of claims 37-40, wherein R⁴ is fluoro.

42. The process of any of claims 37-40, wherein R⁴ is OR⁵, and wherein R⁵ is cyclopropyl.

43. The process of any of claims 37-42, wherein R⁹ is C₁-C₆ alkyl.

44. The process of claim 43, wherein R⁹ is methyl.

45. The process of any of claims 37-44, wherein R¹⁰ is hydrogen.

46. The process of any of claims 37-45, wherein R¹¹ is iodo.

47. The process of any of claims 37-46, wherein step (a) further comprises treating the compound of Formula (I) with an activating reagent, followed by reacting with the amine to provide the compound of Formula (IIA).

48. The process of claim 47, wherein the activating reagent is carbonyl diimidazole (CDI) or carbonyl dichloride.

49. The process of any of claims 37-48, wherein in step (a), the compound of Formula (I) is prepared by treating a compound of Formula (IA) 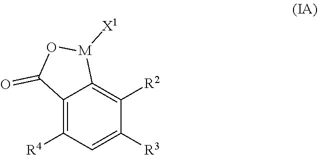
with an electrophile which is I₂, Br₂, Cl₂, an aryl compound, or a heteroaryl compound,
wherein:
M represents a divalent metal of a divalent metal base; and
X¹ represents a counterion of the divalent metal base.

50. The process of claim 49, wherein the compound of Formula (IA) is prepared by treating a compound of Formula (IB) 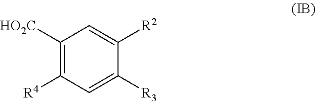
with the divalent metal base M-X¹.

51. The process of any of claims 37-50, wherein R¹ is iodo.

52. The process of any of claims 37-51, wherein the compound of Formula (II) is 4-chloro-3,6-difluoro-2-(4-iodo-1-methyl-1H-pyrazol-5-yl)benzonitrile, which is prepared by:
(a) reacting 4-chloro-3,6-difluoro-2-iodobenzoic acid with an amine to provide 4-chloro-3,6-difluoro-2-iodobenzamide;
(b) dehydrating 4-chloro-3,6-difluoro-2-iodobenzamide to provide 4-chloro-3,6-difluoro-2-iodobenzonitrile;
(c) coupling 4-chloro-3,6-difluoro-2-iodobenzonitrile with an organoboron compound or an organozinc compound to provide 4-chloro-3,6-difluoro-2-(1-methyl-1H-pyrazol-5-yl)benzonitrile; and
(d) iodinating 4-chloro-3,6-difluoro-2-(1-methyl-1H-pyrazol-5-yl)benzonitrile to provide 4-chloro-3,6-difluoro-2-(4-iodo-1-methyl-1H-pyrazol-5-yl)benzonitrile.

53. The process of claim 52, wherein step (a) further comprises treating 4-chloro-3,6-difluoro-2-iodobenzoic acid with an activating reagent, followed by reacting with the amine to provide 4-chloro-3,6-difluoro-2-iodobenzamide.

54. The process of claim 53, wherein the activating reagent is CDI or carbonyl dichloride.

55. The process of any of claims 52-54, wherein in step (a), 4-chloro-3,6-difluoro-2-iodobenzoic acid is prepared by:
(1) treating 4-chloro-2,5-difluorobenzoic acid with a divalent metal base to provide a mixture; and
(2) treating the mixture with iodine to provide 4-chloro-3,6-difluoro-2-iodobenzoic acid.

56. The process of any of claims 37-55, wherein in step (a), the amine is ammonia or ammonium hydroxide.

57. The process of any of claims 37-56, wherein step (a) is carried out in a solution of THE or 2-MeTHF.

58. The process of any of claims 37-57, wherein step (a) is carried out under an inert atmosphere at a temperature in a range of 20 to 60° C.

59. The process of any of claims 37-58, wherein step (b) is carried out in the presence of trifluoroacetic anhydride (TFAA), phosphoryl chloride (POCl₃), thionyl chloride (SOCl₂), or oxalyl chloride (COCl)₂.

60. The process of any of claims 37-59, wherein step (b) is carried out in a solution of THF or 2-MeTHF.

61. The process of any of claims 37-60, wherein step (b) is carried out under an inert atmosphere at a temperature in a range of 0 to 25° C.

62. The process of any of claims 37-61, wherein in step (c), the organoboron compound is a boronic acid or ester having a structure of Formula (IID) 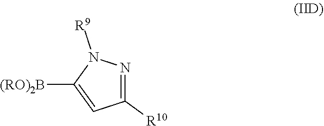
wherein R is hydrogen or C₁-C₆ alkyl.

63. The process of claim 62, wherein the organoboron compound is

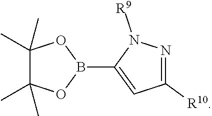

64. The process of any of claims 37-61, wherein in step (c), the organozinc compound has a structure of Formula (IIE) 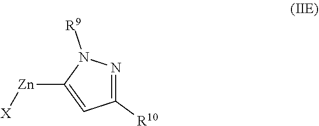
wherein X is I, Br, or Cl.

65. The process of any of claims 37-64, wherein step (c) is carried out in the presence of a catalyst.

66. The process of claim 65, wherein the catalyst is a palladium catalyst.

67. The process of claim 66, wherein the palladium catalyst is [P(t-Bu)₂(n-Bu)]—Pd-G3, Pd(amphos)Cl, or (amphos)₂PdCl₂.

68. The process of claim 66, wherein the palladium catalyst is a pre-catalyst prepared from a palladium source with a ligand.

69. The process of claim 68, wherein the palladium source is Pd(OAc)₂ or Pd₂(dba)₃.

70. The process of claim 68 or claim 69, wherein the ligand is amphos or P(tBu)₂(nBu).

71. The process of claim 65, wherein the catalyst is a nickel catalyst.

72. The process of any of claims 37-71, wherein step (c) is carried out in a solution of THF or toluene.

73. The process of any of claims 37-72, wherein step (c) is carried out under an inert atmosphere at a temperature in a range of 60 to 80° C.

74. The process of any of claims 37-73, wherein step (d) comprises iodinating the compound of step (c) to provide the compound of Formula (II).

75. The process of any of claims 37-74, wherein step (d) is carried out in the presence of an iodinating reagent.

76. The process of claim 75, wherein the iodinating reagent is molecular iodine or N-iodosuccinimide (NIS).

77. The process of any of claims 37-76, wherein step (d) is carried out in a solution of acetonitrile.

78. The process of any of claims 37-77, wherein step (d) is carried out under an inert atmosphere at a temperature in a range of 40 to 60° C.

79. A process for preparing a compound of Formula (III) 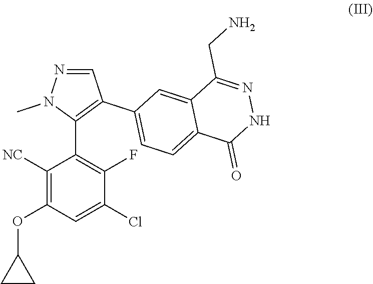 [SMILES: Cn1ncc(-c2ccc3c(=O)[nH]nc(CN)c3c2)c1-c1c(F)c(Cl)cc(OC2CC2)c1C#N]
the process comprising:
(a) treating 4-chloro-2,5-difluorobenzoic acid with a divalent metal base to provide a mixture;
(b) treating the mixture with iodine to provide 4-chloro-3,6-difluoro-2-iodobenzoic acid;
(c) reacting the 4-chloro-3,6-difluoro-2-iodobenzoic acid with an amine to provide 4-chloro-3,6-difluoro-2-iodobenzamide;
(d) dehydrating the 4-chloro-3,6-difluoro-2-iodobenzamide to provide 4-chloro-3,6-difluoro-2-iodobenzonitrile;
(e) coupling the 4-chloro-3,6-difluoro-2-iodobenzonitrile with an organoboron compound or an organozinc compound to provide 4-chloro-3,6-difluoro-2-(1-methyl-1H-pyrazol-5-yl)benzonitrile;
(f) iodinating the 4-chloro-3,6-difluoro-2-(1-methyl-1H-pyrazol-5-yl)benzonitrile to provide 4-chloro-3,6-difluoro-2-(4-iodo-1-methyl-1H-pyrazol-5-yl)benzonitrile; and
(g) reacting the 4-chloro-3,6-difluoro-2-(4-iodo-1-methyl-1H-pyrazol-5-yl)benzonitrile with cyclopropanol to provide 4-chloro-6-cyclopropoxy-3-fluoro-2-(4-iodo-1-methyl-1H-pyrazol-5-yl)benzonitrile.

80. The process of claim 79, wherein the divalent metal base is TMPMgCl·LiCl, (TMP)₂Mg·2LiCl, or i-PrMgCl·LiCl, and wherein TMP is 2,2,6,6-tetramethylpiperidyl.

81. The process of claim 79, wherein the divalent metal base is (TMP)₂Zn·2LiCl or TMPZnCl·LiCl, and wherein TMP is 2,2,6,6-tetramethylpiperidyl.

82. The process of claim 79, wherein the divalent metal base is dialkylzinc or dialkylzinc halide.

83. The process of claim 82, wherein the divalent metal base is Et₂Zn or EtZnCl.

84. The process of any of claims 79-83, wherein step (a) is carried out in a solution of 2-MeTHF under an inert atmosphere at about −20° C.

85. The process of any of claims 79-84, wherein step (b) is carried out in a solution of 2-MeTHE under an inert atmosphere at about −15° C.

86. The process of any of claims 79-85, wherein in step (b), the iodine is used as a solution of I₂ in 2-MeTHF.

87. The process of any of claims 79-86, wherein step (b) further comprises treating the 4-chloro-3,6-difluoro-2-iodobenzoic acid with CDI, followed by treating with the amine in step (c).

88. The process of any of claims 79-87, wherein the amine is ammonia or ammonium hydroxide.

89. The process of any of claims 79-88, wherein in step (e), the organoboron compound is 1-methyl-5-(4,4,5,5-tetramethyl-1,3,2-dioxaborolan-2-yl)-1H-pyrazole.

90. The process of any of claims 79-89, wherein in step (f), the iodinating is carried out with NIS.

91. The process of any of claims 79-90, wherein the process further comprises:
(h) coupling the 4-chloro-6-cyclopropoxy-3-fluoro-2-(4-iodo-1-methyl-1H-pyrazol-5-yl)benzonitrile with a protected organoboron phthalazine compound; and
(i) deprotecting the product of step (h).

92. The process of claim 91, wherein the protected organoboron phthalazine compound is tert-butyl ((4-oxo-7-(4,4,5,5-tetramethyl-1,3,2-dioxaborolan-2-yl)-3,4-dihydrophthalazin-1-yl)methyl)carbamate.

93. The process of claim 92, wherein the protected organoboron phthalazine compound is prepared by treating tert-butyl ((7-chloro-4-oxo-3,4-dihydrophthalazin-1-yl)methyl)carbamate with 4,4,4′,4′,5,5,5′,5′-octamethyl-2,2′-bi(1,3,2-dioxaborolane) in the presence of tris(dibenzylideneacetone)dipalladium (0).

94. The process of claim 93, wherein the preparation of the protected organoboron phthalazine compound is carried out in the presence of potassium acetate.

95. The process of claim 93 or claim 94, wherein the preparation of the protected organoboron phthalazine compound is carried out in a solution of 1,4-dioxane.

96. The process of any of claims 93-95, wherein the preparation of the protected organoboron phthalazine compound is carried out under an inert atmosphere at about 100° C.

97. A compound of Formula (IV) 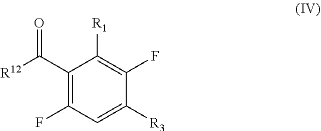
wherein:
R¹ is iodo, an aryl group, or a heteroaryl group;
R³ is bromo or chloro; and
R¹² is-OR¹³ or —NR¹⁴R¹⁵, wherein each of R¹³, R¹⁴, and R¹⁵ is independently hydrogen, C₁-C₆ alkyl, or C₃-C₆ cycloalkyl.

98. The compound of claim 97, wherein R¹ is iodo.

99. The compound of claim 97, wherein R¹ is an aryl group.

100. The compound of claim 99, wherein R¹ is a C₆-C₁₀ aryl group optionally substituted with one or more of C₁-C₆ alkyl, halo C₁-C₆ alkyl, C₃-C₆ cycloalkyl, or C₁-C₆ alkoxy.

101. The compound of claim 99 or claim 100, wherein the aryl group is phenyl, naphthyl, anthracenyl, fluorenyl, benzyl, phenethyl, or naphthylmethyl.

102. The compound of claim 97, wherein R¹ is a heteroaryl group.

103. The compound of claim 102, wherein R¹ is a 5- or 6-member heteroaryl group optionally substituted with one or more of C₁-C₆ alkyl, halo C₁-C₆ alkyl, C₃-C₆ cycloalkyl, or C₁-C₆ alkoxy.

104. The compound of claim 102 or claim 103, wherein the heteroaryl group is furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, or triazinyl.

105. The compound of any of claims 97-104, wherein R³ is chloro.

106. The compound of any of claims 97-104, wherein R³ is bromo.

107. The compound of any of claims 97-106, wherein R¹² is-OH.

108. The compound of any of claims 97-106, wherein R¹² is —NH₂.

109. A composition comprising a mixture of 4-chloro-2,5-difluorobenzoic acid and 4-chloro-3,6-difluoro-2-iodobenzoic acid.

110. The composition of claim 109, wherein the mixture is substantially free of 4-chloro-2,5-difluoro-3-iodobenzoic acid.

111. The composition of claim 109, wherein the mixture comprises greater than about 90% (w/w) of 4-chloro-3,6-difluoro-2-iodobenzoic acid and less about 10% of 4-chloro-2,5-difluoro-3-iodobenzoic acid based on the combined weight of 4-chloro-3,6-difluoro-2-iodobenzoic acid and 4-chloro-2,5-difluoro-3-iodobenzoic acid.

112. The composition of any of claims 109-111, wherein the mixture comprises 4-chloro-3,6-difluoro-2-iodobenzoic acid and 4-chloro-2,5-difluoro-3-iodobenzoic acid in a ratio of 99:1.

113. The composition of any of claims 109-112, wherein the composition further comprises a divalent base.

114. The composition of claim 113, wherein the divalent base is TMPMgCl·LiCl, (TMP)₂Mg·2LiCl, or i-PrMgCl·LiCl, and wherein TMP is 2,2,6,6-tetramethylpiperidyl.

115. The composition of claim 113, wherein the divalent metal base is (TMP)₂Zn·2LiCl or TMPZnCl·LiCl, and wherein TMP is 2,2,6,6-tetramethylpiperidyl.

116. The composition of claim 113, wherein the divalent metal base is dialkylzinc or dialkylzinc halide.

117. The composition of claim 116, wherein the divalent metal base is Et₂Zn or EtZnCl.

118. The composition of any of claims 109-117, wherein the composition further comprises iodine.
